(19) United States
(12) Patent Application Publication
(10) Pub. No.: US 2026/0347310 A1
(43) Pub. Date: Nov. 5, 2026

(54) PHENALKYLAMINES AND METHODS OF MAKING AND USING THE SAME

(71) Applicant: GILGAMESH PHARMACEUTICALS, INC., New York, NY (US)

(72) Inventor: Andrew Carry KRUEGEL, Millington, NJ (US)

(73) Assignee: GILGAMESH PHARMACEUTICALS, INC., New York, NY (US)

(21) Appl. No.: 19/111,234

(22) PCT Filed: Sep. 12, 2023
(86) PCT No.: PCT/US2023/032570

§ 371 Date: Mar. 12, 2025

Related U.S. Application Data
(60) Provisional application No. 63/405,782, filed on Sep. 12, 2022.

(51) Int. Cl. C07C 217/60; A61K 31/137

(52) U.S. Cl. CPC C07C 217/60; A61K 31/137; C07B 2200/05; C07B 2200/07

(57) ABSTRACT

The present disclosure provides phenalkylamine compounds and their use in treating medical disorders, such as psychiatric diseases and disorders. Pharmaceutical compositions and methods of making various phenalkylamine compounds are provided. Also disclosed herein are methods of treating a psychiatric disease or disorder comprising administering to a patient in need thereof a therapeutically effective amount of any of the therapeutic compounds described herein or a pharmaceutical composition containing the therapeutic compound.

### BACKGROUND

Psychiatric illnesses, including depression and anxiety, represent a serious detriment to health and effective human functioning worldwide. Although a number of psychiatric medications are available and extensively prescribed, they fail to deliver relief for many individuals. For those patients who do respond, changes in mood and behavior are often slow to manifest. In recent years, these persistent unmet needs for improved pharmacotherapies to treat psychiatric disorders have led to consideration of previously maligned options. For example, classical serotonergic hallucinogens such as lysergic acid diethylamide (LSD), psilocybin, and dimethyltryptamine (DMT), have been considered as experimental therapeutics for a variety of psychiatric indications.

However, such compounds induce profound hallucinogenic effects, which inhibit normal functioning of individuals so treated. Accordingly, these compounds are currently classified as Schedule I drugs under the Controlled Substances Act due to their high abuse potential, no accepted medical use, and lack of established safety. Such effects are mediated largely through engagement of serotonin receptors. Of particular importance is agonism of the serotonin 2A receptor (5-HT2A), which is responsible for the problematic hallucinogenic activity of these compounds but also thought to be critical for their purported therapeutic effects. Accordingly, compounds of this type that would deliver therapeutic benefits while limiting hallucinogenic activity, and therefore the potential for abuse and adverse events, would be of high therapeutic value.

### SUMMARY

The present disclosure provides, for example, therapeutic compounds which are modulators of 5-HT2A receptors (5-HT2A), their use as medicinal agents, processes for their preparation, and pharmaceutical compositions containing them as an active ingredient both alone or in combination with other agents. It further provides for their use as medicaments and/or in the manufacture of medicaments for the activation of 5-HT2A in warm-blooded animals, such as humans. In particular, this disclosure relates to compounds useful for the treatment of psychiatric diseases or disorders, such as depressive disorders, substance-related disorders, substance-use disorders, and anxiety disorders. The present disclosure also relates to compounds useful for the treatment of headache or headache disorders, inflammatory diseases or disorders, and high intraocular pressure. Further, this disclosure provides compounds that induce useful therapeutic effects while exhibiting attenuated or no hallucinogenic effects. Also provided are pharmaceutical compositions comprising at least one disclosed compound and a pharmaceutically acceptable carrier.

In a first set of embodiments, the therapeutic compound has the following R-enantiomeric structure:

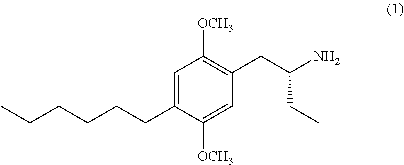 [SMILES: CCCCCCc1cc(OC)c(C[C@H](N)CC)cc1OC]

In some embodiments, the compound of Formula (1) is in enantiomerically pure form. In other embodiments, the compound of Formula (1) is present in a composition which includes the S-enantiomeric compound of Formula (1), provided that the R-enantiomeric compound of Formula (1) is in enantiomeric excess. Pharmaceutically acceptable salts of the compound of Formula (1) are also within the scope of Formula (1).

In a second set of embodiments, the therapeutic compound has the following structure:

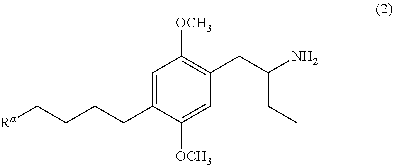

wherein: R^{a} is selected from the group consisting of —CH₂CF₃, —CF₂CH₃, and —CF₂CF₃; and pharmaceutically acceptable salts thereof.

In a third set of embodiments, the therapeutic compound has the following structure:

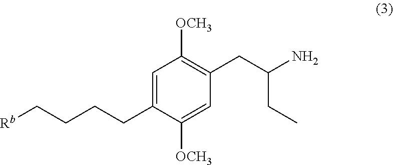

wherein: R^{b} is —CH₂CD₃ or -CD₂CD₃; and pharmaceutically acceptable salts thereof. In embodiments of Formula (3), one or more D in R^{b} is present in greater than the natural abundance, or one or more D in R^{b} is at least or greater than 50% enriched, or one or more D in R^{b} is at least or greater than 90% enriched, or one or more D in R^{b} is at least or greater than 95% enriched.

### BRIEF DESCRIPTION OF THE DRAWING

The file of this patent or application contains at least one drawing executed in color. Copies of this patent or application publication with color drawing(s) will be provided by the Patent and Trademark Office upon request and payment of the necessary fee.

FIG. 1 is an ORTEP crystallographic structure of Compound 1R hydrochloride (ENT-1 2.458 min hydrochloride), where carbon atoms are labeled gray, oxygen atoms are labeled red, the nitrogen atom is labeled blue, and the chlorine atom is labeled green.

### DETAILED DESCRIPTION

The features and other details of the disclosure will now be more particularly described. Before further description of the present disclosure, certain terms employed in the specification, examples and appended claims are collected here. These definitions should be read in light of the remainder of the disclosure and as understood by a person of skill in the art. Unless defined otherwise, all technical and scientific terms used herein have the same meaning as commonly understood by a person of ordinary skill in the art to which this disclosure belongs. Although methods and materials similar or equivalent to those described herein can be used in the practice or testing of embodiments of the present disclosure, suitable methods and materials are described below. All publications, patent applications, patents, and other references mentioned herein are incorporated by reference in their entirety, unless a particular passage is cited. In case of conflict, the present specification, including definitions, will control. In addition, the materials, methods, and examples are illustrative only and not intended to be limiting.

### Definitions

Unless indicated to the contrary, all percentages are by weight.

“Treating” includes any effect, e.g., lessening, reducing, modulating, or eliminating, that results in the improvement of the condition, disease, disorder and the like.

The terms “pharmaceutically acceptable” or “pharmacologically acceptable” include molecular entities and compositions that do not produce an adverse, allergic, or other untoward reaction when administered to an animal, or a human, as appropriate. For human administration, preparations should meet sterility, pyrogenicity, and general safety and purity standards as required by the relevant governmental regulatory body. In embodiments, the term refers to molecular entities and compositions approved by a regulatory agency of the federal or a state government, such as the GRAS list under sections 204(s) and 409 of the Federal Food, Drug and Cosmetic Act, that is subject to premarket review and approval by the FDA or similar lists, such as the U.S. Pharmacopeia or another generally recognized pharmacopeia for use in animals, and more particularly in humans.

The terms “pharmaceutically acceptable carrier” or “pharmaceutically acceptable excipient” as used herein refer to any and all solvents, dispersion media, coatings, isotonic and absorption delaying agents, and the like, that are compatible with pharmaceutical administration. The use of such media and agents for pharmaceutically active substances is well known in the art. The compositions may also contain other active compounds providing supplemental, additional, or enhanced therapeutic functions.

The term “pharmaceutical composition” as used herein refers to a composition comprising at least one compound as disclosed herein formulated together with one or more pharmaceutically acceptable carriers.

The term “pharmaceutically acceptable salt(s)” as used herein refers to salts of acidic or basic groups that may be present in compounds used in the compositions. Compounds included in the present compositions that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmaceutically acceptable anions, including, but not limited to, malate, oxalate, hydrochloride, hydrobromide, hydroiodide, nitrate, sulfate, bisulfate, phosphate, acid phosphate, isonicotinate, acetate, lactate, salicylate, citrate, tartrate, oleate, tannate, pantothenate, bitartrate, ascorbate, succinate, maleate, gentisinate, fumarate, gluconate, glucaronate, saccharate, formate, benzoate, glutamate, methanesulfonate, ethanesulfonate, benzenesulfonate, p-toluenesulfonate and pamoate (i.e., 1,1′-methylene-bis-(2-hydroxy-3-naphthoate)) salts. Compounds included in the present compositions that are acidic in nature are capable of forming a wide variety of salts with various inorganic and organic bases. The bases that may be used to prepare pharmaceutically acceptable base salts of such acidic compounds are those that form non-toxic base salts, i.e., salts containing pharmaceutically acceptable cations. Examples of such salts include alkali metal or alkaline earth metal salts, particularly calcium, magnesium, sodium, lithium, zinc, potassium, and iron salts. Compounds included in the present compositions that include a basic or acidic moiety may also form pharmaceutically acceptable salts with various amino acids. The compounds of the disclosure may contain both acidic and basic groups; for example, one amino and one carboxylic acid group. In such a case, the compound can exist as an acid addition salt, a zwitterion, or a base salt.

“Individual,” “patient,” or “subject” are used interchangeably and include any animal, including mammals, preferably mice, rats, other rodents, rabbits, dogs, cats, swine, cattle, sheep, horses, or primates, and most preferably humans. The compounds of the disclosure can be administered to a mammal, such as a human, but can also be administered to other mammals such as an animal in need of veterinary treatment, e.g., domestic animals (e.g., dogs, cats, and the like), farm animals (e.g., cows, sheep, pigs, horses, and the like) and laboratory animals (e.g., rats, mice, guinea pigs, and the like). The mammal treated in the methods of the disclosure is desirably a mammal in which treatment of a psychiatric disease or disorder is desired.

“Modulation” includes antagonism (e.g., inhibition), agonism, partial antagonism, and/or partial agonism.

In the present specification, the term “therapeutically effective amount” means the amount of the subject compound that will elicit the biological or medical response of a tissue, system or animal, (e.g. mammal or human) that is being sought by the researcher, veterinarian, medical doctor or other clinician. The compounds of the disclosure are administered in therapeutically effective amounts to treat a disease. Alternatively, a therapeutically effective amount of a compound is the quantity required to achieve a desired therapeutic and/or prophylactic effect, such as an amount which results in a decrease in symptoms of a psychiatric disorder, including, but not limited to, reducing the frequency or severity of sadness or lethargy, depressed mood, anxious or sad feelings, diminished interest in all or nearly all activities, significant increased or decreased appetite leading to weight gain or weight loss, insomnia, irritability, fatigue, feelings of worthlessness, feelings of helplessness, inability to concentrate, and recurrent thoughts of death or suicide; or providing a desired pharmacologic and/or physiologic effect, for example, reducing, inhibiting, or reversing one or more of the underlying pathophysiological mechanisms underlying the neurological dysfunction, modulating dopamine levels or signaling, modulating serotonin levels or signaling, modulating norepinephrine levels or signaling, modulating glutamate or GABA levels or signaling, modulating synaptic connectivity or neurogenesis in certain brain regions, or a combination thereof. The precise dosage will vary according to a variety of factors, such as subject-dependent variables (e.g., age, immune system health, clinical symptoms, etc.), the disease or disorder being treated, as well as the route of administration and the pharmacokinetics of the agent being administered.

The compounds of the disclosure may contain one or more chiral centers and, therefore, exist as stereoisomers. The term “stereoisomers” when used herein refers to all enantiomers or diastereomers. Specific stereoisomers of such compounds may be designated by the symbols “(+),” “(−),” “R” or “S,” depending on the configuration of substituents around the stereogenic carbon atom, but the skilled artisan will recognize that a structure may denote a chiral center implicitly. The present disclosure encompasses various stereoisomers of these compounds in pure form and mixtures thereof. Mixtures of enantiomers or diastereomers may be designated “(+)” in nomenclature, but the skilled artisan will recognize that a structure may denote a chiral center implicitly.

The compounds of the disclosure may contain one or more double bonds and, therefore, exist as geometric isomers resulting from the arrangement of substituents around a carbon-carbon double bond. The symbol [symbol] denotes a bond that may be a single, double or triple bond as described herein. Substituents around a carbon-carbon double bond are designated as being in the “Z” or “E” configuration wherein the terms “Z” and “E” are used in accordance with IUPAC standards. Unless otherwise specified, structures depicting double bonds encompass both the “E” and “Z” isomers. Substituents around a carbon-carbon double bond alternatively can be referred to as “cis” or “trans,” where “cis” represents substituents on the same side of the double bond and “trans” represents substituents on opposite sides of the double bond.

Compounds of the disclosure may contain a carbocyclic or heterocyclic ring and therefore, exist as geometric isomers resulting from the arrangement of substituents around the ring. The arrangement of substituents around a carbocyclic or heterocyclic ring are designated as being in the “Z” or “E” configuration wherein the terms “Z” and “E” are used in accordance with IUPAC standards. Unless otherwise specified, structures depicting carbocyclic or heterocyclic rings encompass both “Z” and “E” isomers. Substituents around a carbocyclic or heterocyclic rings may also be referred to as “cis” or “trans”, where the term “cis” represents substituents on the same side of the plane of the ring and the term “trans” represents substituents on opposite sides of the plane of the ring. Mixtures of compounds wherein the substituents are disposed on both the same and opposite sides of the plane of the ring are designated “cis/trans.”

Individual enantiomers and diastereomers of compounds of the present disclosure can be prepared synthetically from commercially available starting materials that contain asymmetric or stereogenic centers, or by preparation of racemic mixtures followed by resolution methods well known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary, (2) salt formation employing an optically active resolving agent, (3) direct separation of the mixture of optical enantiomers on chiral liquid chromatographic columns, or (4) kinetic resolution using stereoselective chemical or enzymatic reagents. Racemic mixtures can also be resolved into their component enantiomers by well-known methods, such as chiral-phase liquid chromatography or crystallizing the compound in a chiral solvent. Stereoselective syntheses, a chemical or enzymatic reaction in which a single reactant forms an unequal mixture of stereoisomers during the creation of a new stereocenter or during the transformation of a pre-existing one, are well known in the art. Stereoselective syntheses encompass both enantio- and diastereoselective transformations, and may involve the use of chiral auxiliaries. For examples, see Carreira and Kvaerno, Classics in Stereoselective Synthesis, Wiley-VCH: Weinheim, 2009.

In some embodiments, a composition prepared herein may be enriched in a specific enantiomer of any compound disclosed herein relative to the corresponding opposite enantiomer of that compound, such that the mixture is not racemic. In such cases, the subject mixture of isomers is understood to have an enantiomeric excess and optical purity >0%. The enantiomeric excess or optical purity of the isomeric mixture may be, for example, >0%, >5%, >25%, >50%, >75%, >90%, >95%, >97%, >98%, or >99%. The enantiomeric excess or optical purity of the isomeric mixture may be, for example, 5-100%, 25-100%, 50-100%, 75-100%, 90-100%, 95-100%, 97-100%, 98-100%, or 99-100%. Thus, for example, contemplated herein is a composition including the S enantiomer of a compound substantially free of the R enantiomer, or the R enantiomer substantially free of the S enantiomer. Further, if the named compound includes more than one chiral center, the scope of the present disclosure also includes compositions containing the various stereoisomers and diastereomers, including mixtures of varying proportions between the various stereoisomers and/or diastereomers or pharmaceutically acceptable salts thereof, as well as compositions including one or more stereoisomers and diastereomers substantially free of one or more of the other stereoisomers and/or diastereomers, respectively. By “substantially free” in this context, it is meant that the composition includes less than, for example, 50%, 25%, 15%, 10%, 8%, 5%, 3%, 2%, or 1% of the minor enantiomer or diastereomer(s). For example, the expression that a compound is enantiomerically pure refers to the compound being substantially free of other stereoisomers, including any other enantiomers or diastereomers.

For clarity, in the context of the present disclosure, chemical structures of a compound depicted with a specific stereochemical orientation at any particular chiral center, as defined by wedge and dash notation, are intended to represent the specified stereoisomer of said compound in substantially pure form, or a mixture enriched in the stereoisomer(s) with the specified stereochemical orientation at the defined chiral center over the stereoisomer(s) with the opposite orientation at said chiral center.

The compounds disclosed herein can exist in solvated as well as unsolvated forms with pharmaceutically acceptable solvents such as water, ethanol, and the like, and it is intended that the disclosure embrace both solvated and unsolvated forms. In one embodiment, the compound is amorphous. In one embodiment, the compound is a single polymorph. In another embodiment, the compound is a mixture of polymorphs. In another embodiment, the compound is in a crystalline form.

The disclosure also embraces isotopically labeled compounds of the disclosure which are identical to those recited herein, except that one or more atoms are replaced by an atom having an atomic mass or mass number different from the atomic mass or mass number usually found in nature. Examples of isotopes that can be incorporated into compounds of the disclosure include isotopes of hydrogen, carbon, nitrogen, oxygen, phosphorus, sulfur, fluorine, and chlorine, such as ²H, ³H, ¹³C, ¹⁴C, ¹⁵N, ¹⁸O, ¹⁷O, ³¹P, ³²p, ³⁵S, ¹⁸F, and ³⁶Cl, respectively. For example, a compound of the disclosure may have one or more H atoms replaced with deuteriums.

Certain isotopically-labeled disclosed compounds (e.g., those labeled with ³H and ¹⁴C) are useful in compound and/or substrate tissue distribution assays. Compounds incorporating tritium (i.e., ³H) or carbon-14 (i.e., ¹⁴C) isotopes are particularly preferred for their ease of preparation and detectability. Further, due to the kinetic isotope effect, substitution with heavier isotopes such as deuterium (i.e., ²H) may afford certain therapeutic advantages resulting from greater metabolic stability (e.g., increased in vivo half-life, reduced dosage requirements, or attenuation of the formation of one or more metabolites) and hence may be preferred in some circumstances. Isotopically labeled compounds of the disclosure can generally be prepared by following procedures analogous to those disclosed in the examples herein by substituting an isotopically labeled reagent for a non-isotopically labeled reagent.

As used in the chemical structures herein, the symbol “D” indicates a deuterium-enriched H-site wherein the isotopic composition of the site is enriched to a level greater than the natural abundance of deuterium. The level of deuterium enrichment at such a site may thus be, for example, greater than 0.02%, greater than 10%, greater than 20%, greater than 30%, greater than 40%, greater than 50%, greater than 60%, greater than 70%, greater than 80%, greater than 90%, greater than 95%, greater than 97%, greater than 98%, or greater than 99%.

Unless otherwise indicated, all numbers expressing quantities of ingredients, reaction conditions, and so forth, used in the specification and claims, are to be understood as being modified in all instances by the term “about.” Accordingly, unless indicated to the contrary, the numerical parameters set forth in this specification and attached claims are approximations that may vary depending upon the desired properties sought to be obtained by the present disclosure.

The terms “about” or “approximately” as used herein mean within an acceptable error range for the particular value as determined by one of ordinary skill in the art, which will depend in part on how the value is measured or determined, i.e., the limitations of the measurement system. For example, “about” can mean within 3 or more than 3 standard deviations, per the practice in the art. Alternatively, “about” can mean a range of up to 20%, a range of up to 10%, a range of up to 5%, and/or a range of up to 1% of a given value. Alternatively, particularly with respect to biological systems or processes, the term can mean within an order of magnitude, e.g., within 5-fold, or within 2-fold, of a value. “About” and “approximately” are used interchangeably herein.

Unless indicated to the contrary, the terms “drugs” and “medicaments” are synonymous.

As used herein, the terms “comprises,” “comprising,” “includes,” “including,” “has,” “having,” or any other variation thereof, are intended to cover a non-exclusive inclusion. For example, a composition, process, method, article, or apparatus that comprises a list of elements is not necessarily limited to only those elements, but may include other elements not expressly listed or inherent to such composition, process, method, article, or apparatus.

Also, the use of “a” or “an” are employed to describe elements and components described herein. This is done merely for convenience and to give a general sense of the scope of the invention. This description should be read to include one or at least one.

Moreover, the singular also includes the plural and vice versa unless it is obvious that it is meant otherwise.

Further, unless expressly stated to the contrary, “or” refers to an inclusive “or” and not to an exclusive “or.” For example, a condition A or B is satisfied by any one of the following: A is true (or present), and B is false (or not present), A is false (or not present), and B is true (or present), and both A and B are true (or present).

Moreover, the term “and/or” is synonymous with the term “or”, as used herein.

When a range or list of values is expressed, an embodiment includes the endpoint of the ranges and/or list and all the points therebetween. For example, a range of 6 to 9, includes the value 6 and 9 and all values therebetween. Similarly, when values are expressed as approximations, by use of the antecedent “about,” it will be understood that the values range from about the two endpoints, where “about” is defined as herein described. All ranges are inclusive and combinable. Further, reference to values stated in ranges includes each and every value within that range.

### I. Compounds

In a first set of embodiments, the present disclosure is directed to a therapeutic compound having the following structure:

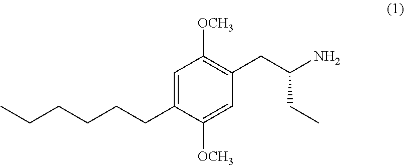 [SMILES: CCCCCCc1cc(OC)c(C[C@H](N)CC)cc1OC]

and pharmaceutically acceptable salts thereof.

In some embodiments, the compound of Formula (1) is in enantiomerically pure (isolated) form. In other embodiments, the compound of Formula (1) is in present in a composition which includes the S-enantiomeric compound of Formula (1), provided that the R-enantiomeric compound of Formula (1) is in enantiomeric excess. By being in enantiomeric excess, the weight ratio of the R-enantiomer to the S-enantiomer is necessarily greater than 1:1, such as at least or greater than 1.1:1, 1.2:1, 1.5:1, 2:1, 3:1, 4:1, 5:1, 10:1, 20:1, 30:1, 50:1, and 100:1. Pharmaceutically acceptable salts of the compound of Formula (1) are also within the scope of Formula (1).

In a second set of embodiments, the present disclosure is directed to fluorinated therapeutic compounds within the following structure:

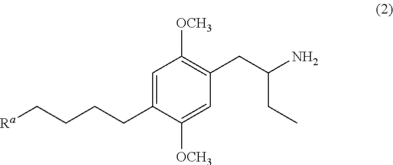

wherein R^{a} is selected from —CH₂CF₃, —CF₂CH₃, and —CF₂CF₃. Pharmaceutically acceptable salts of the compound of Formula (2) are also within the scope of Formula (2).

In one embodiment, the compound of Formula (2) has the following structure:

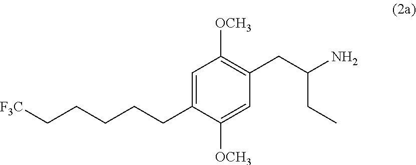 [SMILES: CCC(N)Cc1cc(OC)c(CCCCCC(F)(F)F)cc1OC]

The compound of Formula (2a) may be in racemic form (i.e., a 1:1 mixture of R- and S-enantiomers) or enantiomerically enriched form (i.e., a mixture where one of the enantiomers predominates over the other, as described for the compound of Formula (1)). Pharmaceutically acceptable salts of the compound of Formula (2a) are also within the scope of Formula (2a).

In another embodiment, the compound of Formula (2) has the following structure:

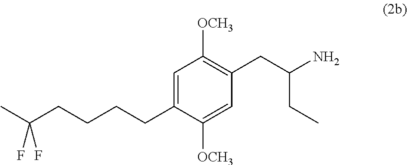 [SMILES: CCC(N)Cc1cc(OC)c(CCCCC(C)(F)F)cc1OC]

The compound of Formula (2b) may be in racemic form (i.e., a 1:1 mixture of R- and S-enantiomers) or enantiomerically enriched form (i.e., a mixture where one of the enantiomers predominates over the other, as described for the compound of Formula (1)). Pharmaceutically acceptable salts of the compound of Formula (2b) are also within the scope of Formula (2b).

In another embodiment, the compound of Formula (2) has the following structure:

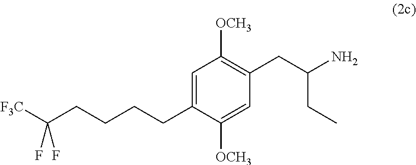 [SMILES: CCC(N)Cc1cc(OC)c(CCCCC(F)(F)C(F)(F)F)cc1OC]

The compound of Formula (2c) may be in racemic form (i.e., a 1:1 mixture of R- and S-enantiomers) or enantiomerically enriched form (i.e., a mixture where one of the enantiomers predominates over the other, as described for the compound of Formula (1)). Pharmaceutically acceptable salts of the compound of Formula (2c) are also within the scope of Formula (2c).

In a third set of embodiments, the present disclosure is directed to deuterated therapeutic compounds within the following structure:

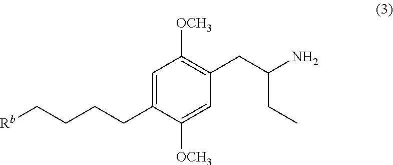

wherein R^{b} is —CH₂CD₃ or -CD₂CD₃; and pharmaceutically acceptable salts thereof. In embodiments of Formula (3), one or more D in R^{b} is present in greater than the natural abundance, or one or more D in R^{b} is at least or greater than 50% enriched, or one or more D in R^{b} is at least or greater than 90% enriched, or one or more D in R^{b} is at least or greater than 95% enriched. In some embodiments, the level of deuterium at each deuterium-enriched H site of the compound is 0.02% to 100%. In some embodiments, the level of deuterium at each deuterium-enriched H site of the compound is 50%-100%, 70%-100%, 90%-100%, 95%-100%, 96%-100%, 97%-100%, 98%-100%, or 99%-100%.

In one embodiment, the compound of Formula (3) has the following structure:

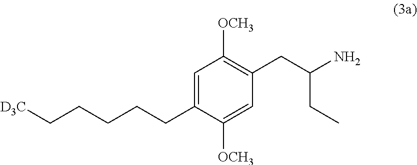 [SMILES: [2H]C([2H])([2H])CCCCCc1cc(OC)c(CC(N)CC)cc1OC]

The compound of Formula (3a) may be in racemic form (i.e., 1:1 mixture of R- and S-enantiomers) or enantiomerically enriched form (i.e., a mixture where one of the enantiomers predominates over the other, as described for the compound of Formula (1)). Pharmaceutically acceptable salts of the compound of Formula (3a) are also within the scope of Formula (3a).

In another embodiment, the compound of Formula (3) has the following structure:

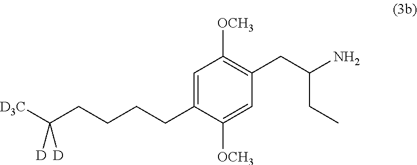 [SMILES: [2H]C([2H])([2H])C([2H])([2H])CCCCc1cc(OC)c(CC(N)CC)cc1OC]

The compound of Formula (3b) may be in racemic form (i.e., 1:1 mixture of R- and S-enantiomers) or enantiomerically enriched form (i.e., a mixture where one of the enantiomers predominates over the other, as described for the compound of Formula (1)). Pharmaceutically acceptable salts of the compound of Formula (3b) are also within the scope of Formula (3b).

Salts of compounds of the present disclosure can be prepared by the reaction of a compound of the present disclosure with an appropriate acid in a suitable solvent, or mixture of solvents (such as an ether, for example, diethyl ether, or an alcohol, for example ethanol, or an aqueous solvent) using conventional procedures. Salts of compounds of the present disclosure can be exchanged for other salts by treatment using conventional ion-exchange chromatography procedures or reformation of the free base followed by reaction with an alternative acid.

Where it is desired to obtain a particular enantiomer of a compound of the present disclosure, this may be produced from a corresponding mixture of enantiomers by employing any suitable conventional procedure for resolving enantiomers. For example, diastereomeric derivatives (such as salts) can be produced by reaction of a mixture of enantiomers of a compound of the present disclosure (such as a racemate) and an appropriate chiral compound (such as a chiral acid). The diastereomers can then be separated by any conventional means such as crystallization, and the desired enantiomer recovered (such as by treatment with a base in the instance where the diastereomer is an acid salt). Alternatively, a racemic mixture of esters can be resolved by kinetic hydrolysis using a variety of biocatalysts (for example, see Patel Stereoselective Biocatalysts, Marcel Decker; New York 2000).

In another resolution process, a racemate of a compound of the present disclosure can be separated using chiral High Performance Liquid Chromatography. Alternatively, a particular enantiomer can be obtained by using an appropriate chiral intermediate in one of the processes described above. Chromatography, recrystallisation, and other conventional separation procedures may also be used with intermediates or final products where it is desired to obtain a particular diastereomer, regioisomer, or geometric isomer of the compounds disclosed herein.

### II. Pharmaceutical Compositions and Kits

Another aspect of the disclosure provides pharmaceutical compositions comprising compounds as disclosed herein formulated together with a pharmaceutically acceptable carrier. In particular, the present disclosure provides pharmaceutical compositions comprising compounds as disclosed herein formulated together with one or more pharmaceutically acceptable carriers. Pharmaceutical compositions typically include a pharmaceutically acceptable carrier in which the one or more active compounds are contained (e.g., dissolved, suspended, or admixed). As well known, the pharmaceutically acceptable carrier is composed of one or more substances that are considered safe and effective. The carrier includes all components present in the pharmaceutical formulation other than the active ingredient(s). The term “carrier” includes, but is not limited to, diluents, binders, lubricants, glidants, disintegrants, fillers, and coating compositions. Suitable dosage forms for a compound disclosed herein include, but are not limited to, oral forms, such as tablets, hard or soft gelatin capsules, powders, granules and oral solutions, syrups or suspensions, troches, as well as sublingual, buccal, intratracheal, intraocular, or intranasal forms, forms adapted to inhalation, topical forms, transdermal forms, or parenteral forms, for example, forms adapted for intravenous, intra-arterial, intraperitoneal, intrathecal, intraventricular, intramuscular or subcutaneous administration. In embodiments, for such parenteral administration, it may be in the form of a sterile aqueous solution which may contain other substances, for example, enough salts or glucose to make the solution isotonic with blood. The aqueous solutions should be suitably buffered (preferably to a pH of from 3 to 9), if necessary. The preparation of suitable parenteral formulations under sterile conditions is readily accomplished by standard pharmaceutical techniques well-known to those skilled in the art.

The pharmaceutical formulations include those suitable for oral, topical, buccal, parenteral (e.g., subcutaneous, intramuscular, intradermal, or intravenous), rectal, vaginal, intranasal, aerosol, or vaporization administration, although the most suitable form of administration in any given case will depend on the degree and severity of the condition being treated and on the nature of the particular compound being used. For example, disclosed compositions may be formulated as a unit dose, and/or may be formulated for oral or subcutaneous administration.

Exemplary pharmaceutical compositions of this disclosure may be used in the form of a pharmaceutical preparation, for example, in solid, semisolid or liquid form, which contains one or more of the compounds of the disclosure, as an active ingredient, in admixture with an organic or inorganic carrier or excipient suitable for external, enteral or parenteral applications. The active ingredient may be compounded, for example, with the usual non-toxic, pharmaceutically acceptable carriers for tablets, pellets, capsules, suppositories, solutions, emulsions, suspensions, and any other form suitable for use. The active object compound is included in the pharmaceutical composition in an amount sufficient to produce the desired effect upon the process or condition of the disease.

For preparing solid compositions such as tablets, the principal active ingredient may be mixed with a pharmaceutical carrier, e.g., conventional tableting ingredients such as corn starch, lactose, sucrose, sorbitol, talc, stearic acid, magnesium stearate, dicalcium phosphate or gums, and other pharmaceutical diluents, e.g., water, to form a solid preformulation composition containing a homogeneous mixture of a compound of the disclosure, or a non-toxic pharmaceutically acceptable salt thereof. When referring to these preformulation compositions as homogeneous, it is meant that the active ingredient is dispersed evenly throughout the composition so that the composition may be readily subdivided into equally effective unit dosage forms such as tablets, pills and capsules.

In solid dosage forms for oral administration (capsules, tablets, pills, dragees, powders, granules and the like), the subject composition is mixed with one or more pharmaceutically acceptable carriers, such as sodium citrate or dicalcium phosphate, and/or any of the following: (1) fillers or extenders, such as starches, lactose, sucrose, glucose, mannitol, and/or silicic acid; (2) binders, such as, for example, carboxymethylcellulose, alginates, gelatin, polyvinyl pyrrolidone, sucrose and/or acacia; (3) humectants, such as glycerol; (4) disintegrating agents, such as agar-agar, calcium carbonate, potato or tapioca starch, alginic acid, certain silicates, and sodium carbonate; (5) solution retarding agents, such as paraffin; (6) absorption accelerators, such as quaternary ammonium compounds; (7) wetting agents, such as, for example, acetyl alcohol and glycerol monostearate; (8) absorbents, such as kaolin and bentonite clay; (9) lubricants, such a talc, calcium stearate, magnesium stearate, solid polyethylene glycols, sodium lauryl sulfate, and mixtures thereof; and (10) coloring agents. In the case of capsules, tablets and pills, the compositions may also comprise buffering agents. Solid compositions of a similar type may also be employed as fillers in soft and hard-filled gelatin capsules using such excipients as lactose or milk sugars, as well as high molecular weight polyethylene glycols and the like.

A tablet may be made by compression or molding, optionally with one or more accessory ingredients. Compressed tablets may be prepared using binder (for example, gelatin or hydroxypropylmethyl cellulose), lubricant, inert diluent, preservative, disintegrant (for example, sodium starch glycolate or cross-linked sodium carboxymethyl cellulose), surface-active or dispersing agent. Molded tablets may be made by molding in a suitable machine a mixture of the subject composition moistened with an inert liquid diluent. Tablets, and other solid dosage forms, such as dragees, capsules, pills and granules, may optionally be scored or prepared with coatings and shells, such as enteric coatings and other coatings well known in the pharmaceutical-formulating art.

Compositions for inhalation or insufflation include solutions and suspensions in pharmaceutically acceptable, aqueous or organic solvents, or mixtures thereof, and powders. Liquid dosage forms for oral administration include pharmaceutically acceptable emulsions, microemulsions, solutions, suspensions, syrups and elixirs. In addition to the subject composition, the liquid dosage forms may contain inert diluents commonly used in the art, such as, for example, water or other solvents, solubilizing agents and emulsifiers, such as ethyl alcohol, isopropyl alcohol, ethyl carbonate, ethyl acetate, benzyl alcohol, benzyl benzoate, propylene glycol, 1,3-butylene glycol, oils (in particular, cottonseed, groundnut, corn, germ, olive, castor and sesame oils), glycerol, tetrahydrofuryl alcohol, polyethylene glycols and fatty acid esters of sorbitan, cyclodextrins and mixtures thereof.

Suspensions, in addition to the subject composition, may contain suspending agents as, for example, ethoxylated isostearyl alcohols, polyoxyethylene sorbitol and sorbitan esters, microcrystalline cellulose, aluminum metahydroxide, bentonite, agar-agar and tragacanth, and mixtures thereof.

Formulations for rectal or vaginal administration may be presented as a suppository, which may be prepared by mixing a subject composition with one or more suitable non-irritating excipients or carriers comprising, for example, cocoa butter, polyethylene glycol, a suppository wax or a salicylate, and which is solid at room temperature, but liquid at body temperature and, therefore, will melt in the body cavity and release the active agent.

Dosage forms for transdermal administration of a subject composition include powders, sprays, ointments, pastes, creams, lotions, gels, solutions, patches and inhalants. The active component may be mixed under sterile conditions with a pharmaceutically acceptable carrier, and with any preservatives, buffers, or propellants which may be required.

The ointments, pastes, creams and gels may contain, in addition to a subject composition, excipients, such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays may contain, in addition to a subject composition, excipients such as lactose, talc, silicic acid, aluminum hydroxide, calcium silicates and polyamide powder, or mixtures of these substances. Sprays may additionally contain customary propellants, such as chlorofluorohydrocarbons and volatile unsubstituted hydrocarbons, such as butane and propane.

Compositions and compounds of the present disclosure may alternatively be administered by aerosol. This is accomplished by preparing an aqueous aerosol, liposomal preparation or solid particles containing the compound. A non-aqueous (e.g., fluorocarbon propellant) suspension could be used. Sonic nebulizers may be used because they minimize exposing the agent to shear, which may result in degradation of the compounds contained in the subject compositions. Ordinarily, an aqueous aerosol is made by formulating an aqueous solution or suspension of a subject composition together with conventional pharmaceutically acceptable carriers and stabilizers. The carriers and stabilizers vary with the requirements of the particular subject composition, but typically include non-ionic surfactants (Tweens, Pluronics, or polyethylene glycol), innocuous proteins like serum albumin, sorbitan esters, oleic acid, lecithin, amino acids such as glycine, buffers, salts, sugars or sugar alcohols. Aerosols generally are prepared from isotonic solutions.

Pharmaceutical compositions of this disclosure suitable for parenteral administration comprise a subject composition in combination with one or more pharmaceutically-acceptable sterile isotonic aqueous or non-aqueous solutions, dispersions, suspensions or emulsions, or sterile powders which may be reconstituted into sterile injectable solutions or dispersions just prior to use, which may contain antioxidants, buffers, bacteriostats, solutes which render the formulation isotonic with the blood of the intended recipient or suspending or thickening agents.

Examples of suitable aqueous and non-aqueous carriers which may be employed in the pharmaceutical compositions of the disclosure include water, ethanol, polyols (such as glycerol, propylene glycol, polyethylene glycol, and the like), and suitable mixtures thereof, vegetable oils, such as olive oil, and injectable organic esters, such as ethyl oleate and cyclodextrins. Proper fluidity may be maintained, for example, by the use of coating materials, such as lecithin, by the maintenance of the required particle size in the case of dispersions, and by the use of surfactants

In another aspect, the disclosure provides enteral pharmaceutical formulations including a disclosed compound and an enteric material; and a pharmaceutically acceptable carrier or excipient thereof. Enteric materials refer to polymers that are substantially insoluble in the acidic environment of the stomach, and that are predominantly soluble in intestinal fluids at specific pHs. The small intestine is the part of the gastrointestinal tract (gut) between the stomach and the large intestine, and includes the duodenum, jejunum, and ileum. The pH of the duodenum is about 5.5, the pH of the jejunum is about 6.5 and the pH of the distal ileum is about 7.5. Accordingly, enteric materials are not soluble, for example, until a pH of about 5.0, of about 5.2, of about 5.4, of about 5.6, of about 5.8, of about 6.0, of about 6.2, of about 6.4, of about 6.6, of about 6.8, of about 7.0, of about 7.2, of about 7.4, of about 7.6, of about 7.8, of about 8.0, of about 8.2, of about 8.4, of about 8.6, of about 8.8, of about 9.0, of about 9.2, of about 9.4, of about 9.6, of about 9.8, or of about 10.0. Exemplary enteric materials include cellulose acetate phthalate (CAP), hydroxypropyl methylcellulose phthalate (HPMCP), polyvinyl acetate phthalate (PVAP), hydroxypropyl methylcellulose acetate succinate (HPMCAS), cellulose acetate trimellitate, hydroxypropyl methylcellulose succinate, cellulose acetate succinate, cellulose acetate hexahydrophthalate, cellulose propionate phthalate, cellulose acetate maleate, cellulose acetate butyrate, cellulose acetate propionate, copolymer of methylmethacrylic acid and methyl methacrylate, copolymer of methyl acrylate, methylmethacrylate and methacrylic acid, copolymer of methylvinyl ether and maleic anhydride (Gantrez ES series), ethyl methacrylate-methylmethacrylate-chlorotrimethylammonium ethyl acrylate copolymer, natural resins such as zein, shellac and copal collophorium, and several commercially available enteric dispersion systems (e. g., Eudragit L30D55, Eudragit FS30D, Eudragit L100, Eudragit S100, Kollicoat EMM30D, Estacryl 30D, Coateric, and Aquateric). The solubility of each of the above materials is either known or is readily determinable in vitro. The foregoing is a list of possible materials, but one of skill in the art with the benefit of the disclosure would recognize that it is not comprehensive and that there are other enteric materials that would meet the objectives of the present disclosure.

Pharmaceutical compositions herein may also be provided with immediate release, delayed release, extended release, or modified release profiles. In embodiments, pharmaceutical compositions with different drug release profiles may be combined to create a two-phase or three-phase release profile. For example, pharmaceutical compositions may be provided with an immediate release and an extended release profile. In embodiments, pharmaceutical compositions may be provided with an extended release and delayed release profile. Such composition may be provided as pulsatile formulations, multilayer tablets, or capsules containing tablets, beads, granules, or the like.

The pharmaceutical composition may also include one or more auxiliary agents. The auxiliary agent(s), also may also be referred to as accessory ingredient(s), include any of the additional conventional substances of the art, such as fillers, binders, diluents, disintegrants, lubricants, colorants, flavoring agents, anti-oxidants, and wetting agents. Such auxiliary agents are suitably selected with respect to the intended form and route of administration and as consistent with conventional pharmaceutical practices.

The present disclosure also provides kits for use by, e.g., a consumer in need of treatment with a disclosed compound. Such kits include a suitable dosage form such as those described above and instructions describing the method of using such dosage form to treat a medical disorder, for example, a psychiatric disease or disorder. The instructions direct the user to administer the dosage form according to administration modes known to those skilled in the art. Such kits could advantageously be packaged and sold in single or multiple kit units. An example of such a kit is a so-called blister pack. Blister packs are well known in the packaging industry and are being widely used for the packaging of pharmaceutical unit dosage forms (tablets, capsules, and the like). Blister packs generally consist of a sheet of relatively stiff material covered with a foil of a preferably transparent plastic material. During the packaging process recesses are formed in the plastic foil. The recesses have the size and shape of the tablets or capsules to be packed. Next, the tablets or capsules are placed in the recesses and the sheet of relatively stiff material is sealed against the plastic foil at the face of the foil which is opposite from the direction in which the recesses were formed. As a result, the tablets or capsules are sealed in the recesses between the plastic foil and the sheet. Preferably the strength of the sheet is such that the tablets or capsules can be removed from the blister pack by manually applying pressure on the recesses whereby an opening is formed in the sheet at the place of the recess. The tablet or capsule can then be removed via said opening.

It may be desirable to provide a memory aid on the kit, e.g., in the form of numbers next to the tablets or capsules whereby the numbers correspond with the days of the regimen which the tablets or capsules so specified should be ingested. Another example of such a memory aid is a calendar printed on the card, e.g., as follows “First Week, Monday, Tuesday, . . . , etc. . . . Second Week, Monday, Tuesday, . . . , etc”. Other variations of memory aids will be readily apparent. A “daily dose” can be a single tablet or capsule or several pills or capsules to be taken on a given day. Also, a daily dose of a first compound can consist of one tablet or capsule while a daily dose of the second compound can consist of several tablets or capsules and vice versa. The memory aid should reflect this.

Also contemplated herein are methods and compositions that include a second active agent or administering a second active agent.

### III. Methods

Another aspect of the disclosure provides methods of modulating the activity of 5-HT2A. Such methods comprise exposing said receptor to a compound described herein. In some embodiments, the compound utilized by one or more of the foregoing methods is one of the generic, subgeneric, or specific compounds described herein. The ability of compounds described herein to modulate, activate, or inhibit 5-HT2A can be evaluated by procedures known in the art and/or described herein. Another aspect of the disclosure provides methods of treating a disease associated with expression or activity of 5-HT2A in a patient. For example, a contemplated method includes administering a disclosed compound in an amount sufficient to establish activation of 5-HT2A effective to decrease the symptoms of a psychiatric disease or disorder in the patient. Further, treatment with a disclosed compound may also increase neuroplasticity or neurogenesis in a 5-HT2A-dependent manner.

In certain embodiments, the compound utilized by one or more of the foregoing methods is one of the generic, subgeneric, or specific compounds described herein.

In some embodiments, the present disclosure provides a method of treating a psychiatric disease or disorder comprising administering to a patient in need thereof a therapeutically effective amount of a compound of the present disclosure, such as any one of compounds of Formula (1), (2), or (3), or sub-formula thereof.

In some embodiments, the psychiatric disease or disorder is selected from the group consisting of major depressive disorder, persistent depressive disorder, postpartum depression, premenstrual dysphoric disorder, seasonal affective disorder, psychotic depression, disruptive mood dysregulation disorder, substance/medication-induced depressive disorder, and depressive disorder due to another medical condition.

In some embodiments, the psychiatric disease or disorder is selected from the group consisting of bipolar disorder I, bipolar disorder II, cyclothymic disorder, substance/medication-induced bipolar and related disorder, and bipolar and related disorder due to another medical condition.

In some embodiments, the psychiatric disease or disorder is a substance-related disorder or substance-use disorder.

In some embodiments, the psychiatric disease or disorder is selected from the group consisting of separation anxiety disorder, selective mutism, specific phobia, social anxiety disorder, panic disorder, panic attach, agoraphobia, generalized anxiety disorder, substance/medication-induced anxiety disorder, anxiety disorder due to another medical condition.

In some embodiments, the psychiatric disease or disorder is selected from the group consisting of obsessive-compulsive and related disorders, trauma- and stressor-related disorders, feeding and eating disorders, borderline personality disorder, attention-deficit/hyperactivity disorder, and autism spectrum disorder.

In some embodiments, the psychiatric disorder is a neurocognitive disorder.

In some embodiments, the psychiatric disease or disorder is a treatment-resistant disease or disorder, such as treatment-resistant depressive disorder.

The present disclosure further provides a method of enhancing creativity or cognition in a subject, said method comprising administering to said subject a composition comprising an effective amount of a compound of the present disclosure.

In some embodiments, the compounds, methods, and compositions may be used to treat a psychiatric disorder including Depressive Disorders, e.g., Major Depressive Disorder, Persistent Depressive Disorder, Postpartum Depression, Premenstrual Dysphoric Disorder, Seasonal Affective Disorder, Psychotic Depression, Disruptive Mood Dysregulation Disorder, Substance/Medication-Induced Depressive Disorder, and Depressive Disorder Due to Another Medical Condition.

Also provided herein are compounds, methods, and compositions for treating refractory or treatment-resistant depression, e.g., patients suffering from a depressive disorder that does not, and/or has not, responded to adequate courses of at least one, or at least two, other antidepressant compounds or therapeutics. As used herein “depressive disorder” encompasses refractory depression.

In some embodiments, the compounds, methods, and compositions may be used to treat a psychiatric disorder including Bipolar and Related Disorders, e.g., Bipolar I Disorder, Bipolar II Disorder, Cyclothymic Disorder, Substance/Medication-Induced Bipolar and Related Disorder, and Bipolar and Related Disorder Due to Another Medical Condition.

In some embodiments, the compounds, methods, and compositions may be used to treat a psychiatric disorder including Substance-Related Disorders, e.g., preventing a substance use craving, diminishing a substance use craving, and/or facilitating substance use cessation or withdrawal. Substance use disorders involve abuse of psychoactive compounds such as alcohol, caffeine, cannabis, inhalants, opioids, sedatives, hypnotics, anxiolytics, stimulants, nicotine and tobacco. As used herein “substance” or “substances” are psychoactive compounds which can be addictive such as alcohol, caffeine, cannabis, hallucinogens, inhalants, opioids, sedatives, hypnotics, anxiolytics, stimulants, nicotine and tobacco. For example, the methods and compositions may be used to facilitate smoking cessation or cessation of opioid use.

In some embodiments, the compounds, methods, and compositions may be used to treat a psychiatric disorder including Anxiety Disorders, e.g., Separation Anxiety Disorder, Selective Mutism, Specific Phobia, Social Anxiety Disorder (Social Phobia), Panic Disorder, Panic Attack, Agoraphobia, Generalized Anxiety Disorder, Substance/Medication-Induced Anxiety Disorder, and Anxiety Disorder Due to Another Medical Condition.

In some embodiments, the compounds, methods, and compositions may be used to treat a psychiatric disorder including Obsessive-Compulsive and Related Disorders, e.g., Obsessive-Compulsive Disorder, Body Dysmorphic Disorder, Hoarding Disorder, Trichotillomania (Hair-Pulling Disorder), Excoriation (Skin-Picking) Disorder, Substance/Medication-Induced Obsessive-Compulsive and Related Disorder, and Obsessive-Compulsive and Related Disorder Due to Another Medical Condition.

In some embodiments, the compounds, methods, and compositions may be used to treat a psychiatric disorder including Trauma- and Stressor-Related Disorders, e.g., Reactive Attachment Disorder, Disinhibited Social Engagement Disorder, Posttraumatic Stress Disorder, Acute Stress Disorder, and Adjustment Disorders.

In some embodiments, the compounds, methods, and compositions may be used to treat a psychiatric disorder including Feeding and Eating Disorders, e.g., Anorexia Nervosa, Bulimia Nervosa, Binge-Eating Disorder, Pica, Rumination Disorder, and Avoidant/Restrictive Food Intake Disorder.

In some embodiments, the compounds, methods, and compositions may be used to treat a psychiatric disorder including Neurocognitive Disorders, e.g., Delirium, Major Neurocognitive Disorder, Mild Neurocognitive Disorder, Major or Mild Neurocognitive Disorder Due to Alzheimer's Disease, Major or Mild Frontotemporal Neurocognitive Disorder, Major or Mild Neurocognitive Disorder With Lewy Bodies, Major or Mild Vascular Neurocognitive Disorder, Major or Mild Neurocognitive Disorder Due to Traumatic Brain Injury, Substance/Medication-Induced Major or Mild Neurocognitive Disorder, Major or Mild Neurocognitive Disorder Due to HIV Infection, Major or Mild Neurocognitive Disorder Due to Prion Disease, Major or Mild Neurocognitive Disorder Due to Parkinson's Disease, Major or Mild Neurocognitive Disorder Due to Huntington's Disease, Major or Mild Neurocognitive Disorder Due to Another Medical Condition, and Major or Mild Neurocognitive Disorder Due to Multiple Etiologies.

In some embodiments, the compounds, methods, and compositions may be used to treat a psychiatric disorder including Neurodevelopmental Disorders, e.g., Autism Spectrum Disorder, Attention-Deficit/Hyperactivity Disorder, Stereotypic Movement Disorder, Tic Disorders, Tourette's Disorder, Persistent (Chronic) Motor or Vocal Tic Disorder, and Provisional Tic Disorder.

In some embodiments, the compounds, methods, and compositions may be used to treat a psychiatric disorder including Personality Disorders, e.g., Borderline Personality Disorder.

In some embodiments, the compounds, methods, and compositions may be used to treat a psychiatric disorder including Sexual Dysfunctions, e.g., Delayed Ejaculation, Erectile Disorder, Female Orgasmic Disorder, Female Sexual Interest/Arousal Disorder, Genito-Pelvic Pain/Penetration Disorder, Male Hypoactive Sexual Desire Disorder, Premature (Early) Ejaculation, and Substance/Medication-Induced Sexual Dysfunction.

In some embodiments, the compounds, methods, and compositions may be used to treat a psychiatric disorder including Gender Dysphoria, e.g., Gender Dysphoria.

In some embodiments, the compounds, methods, and compositions may be used to treat a headache or a headache disorder. In some embodiments, the headache disorder is a migraine or cluster headaches.

In some embodiments, the compounds, methods, and compositions may be used to treat an inflammatory disorder. In some embodiments, an inflammatory disorder is inflammatory bowel disease, including ulcerative colitis and Crohn's disease. In some embodiments, an inflammatory disorder is inflammatory bowel syndrome. In some embodiments, an inflammatory disorder is an inflammation-related cardiovascular disorder, such as artherosclerosis and coronary artery disease. In some embodiments, an inflammatory disorder is an inflammatory disorder dependent on TNF-α activity.

In some embodiments, the compounds, methods, and compositions may be used to treat high intraocular pressure.

The compounds of the disclosure may be administered to patients (animals and humans) in need of such treatment in dosages that will provide optimal pharmaceutical efficacy. It will be appreciated that the dose required for use in any particular application will vary from patient to patient, not only with the particular compound or composition selected, but also with the route of administration, the nature of the condition being treated, the age and condition of the patient, concurrent medication or special diets then being followed by the patient, and other factors which those skilled in the art will recognize, with the appropriate dosage ultimately being at the discretion of the attendant physician. For treating clinical conditions and diseases noted above, a compound of this disclosure may be administered orally, subcutaneously, topically, parenterally, by inhalation spray, by vaporization, intranasally, or rectally in dosage unit formulations containing conventional non-toxic pharmaceutically acceptable carriers, adjuvants and vehicles. Parenteral administration may include subcutaneous injections, intravenous injections, intramuscular injections, or infusion techniques.

Treatment can be continued for as long or as short a period as desired. The compositions may be administered on a regimen of, for example, one to four or more times per day. A suitable treatment period can be, for example, at least about one week, at least about two weeks, at least about one month, at least about six months, at least about 1 year, or indefinitely. A treatment period can terminate when a desired result, for example a decrease in symptoms of a psychiatric disorder, is achieved. A treatment regimen can include a corrective phase, during which a dose sufficient to provide symptomatic relief is administered, and can be followed by a maintenance phase, during which a lower dose sufficient to prevent a return of symptoms is administered. A suitable maintenance dose is likely to be found in the lower parts of the dose ranges provided herein, but corrective and maintenance doses can readily be established for individual subjects by those of skill in the art without undue experimentation, based on the disclosure herein. Maintenance doses can be employed to maintain remission in subjects whose symptoms have been previously controlled by other means, including treatments employing other pharmacological agents.

In some embodiments, methods include treating a psychiatric disorder, e.g., a depressive disorder, by administering to a patient in need thereof a pharmaceutical composition including about 0.01 mg to about 400 mg of a compound of the present disclosure. In some embodiments, doses may be, e.g., in the range of about 0.01 to 400 mg, 0.01 to 300 mg, 0.01 to 250 mg, 0.01 to 200 mg, 0.01 to 150 mg, 0.01 to 100 mg, 0.01 to 75 mg, 0.01 to 50 mg, 0.01 to 25 mg, 0.01 to 20 mg, 0.01 to 15 mg, 0.01 to 10 mg, 0.01 to 5 mg, 0.01 to 1 mg, 0.01 to 0.5 mg, 0.01 to 0.1 mg, 0.1 to 400 mg, 0.1 to 300 mg, 0.1 to 250 mg, 0.1 to 200 mg, 0.1 to 150 mg, 0.1 to 100 mg, 0.1 to 75 mg, 0.1 to 50 mg, 0.1 to 25 mg, 0.1 to 20 mg, 0.1 to 15 mg, 0.1 to 10 mg, 0.1 to 5 mg, 0.1 to 1 mg, 10 to 400 mg, 10 to 300 mg, 10 to 250 mg, 10 to 200 mg, 10 to 150 mg, 10 to 100 mg, 10 to 50 mg, 10 to 25 mg, 10 to 15 mg, 20 to 400 mg, 20 to 300 mg, 20 to 250 mg, 20 to 200 mg, 20 to 150 mg, 20 to 100 mg, 20 to 50 mg, 50 to 400 mg, 50 to 300 mg, 50 to 250 mg, 50 to 200 mg, 50 to 150 mg, 50 to 100 mg, 100 to 400 mg, 100 to 300 mg, 100 to 250 mg, 100 to 200 mg, with doses of, e.g., about 0.01 mg, 0.025 mg, 0.05 mg, 0.1 mg, 0.15 mg, 0.25 mg, 0.5 mg, 0.75 mg, 1 mg, 1.25 mg, 1.5 mg, 1.75 mg, 2.0 mg, 2.5 mg, 3.0 mg, 3.5 mg, 4.0 mg, 4.5 mg, 5 mg, 10 mg, 15 mg, 20 mg, 25 mg, 30, mg, 35 mg, 40 mg, 45 mg, 50 mg, 75 mg, 100 mg, 125 mg, 150 mg, 175 mg, 200 mg, 225 mg, 250 mg, 275 mg, 300 mg, and 400 mg being examples.

In some embodiments, dosages may include amounts of a compound of the present disclosure or a pharmaceutically acceptable salt thereof in the range of about, e.g., 1 mg to 50 mg, 1 mg to 40 mg, 1 mg to 30 mg, 1 mg to 20 mg, 1 mg to 15 mg, 1 mg to 10 mg, 0.1 mg to 10 mg, 0.1 to 5 mg, or 0.1 to 1 mg, with doses of 0.1 mg, 0.2 mg, 0.3 mg, 0.4 mg, 0.5 mg, 0.6 mg, 0.7 mg, 0.8 mg, 0.9 mg, 1.5 mg, 1.0 mg, 1.75 mg, 2 mg, 2.5 mg, 2.75 mg, 3 mg, 3.5 mg, 3.75 mg, 4 mg, 4.5 mg, 4.75 mg, 5 mg, 5.5 mg, 6 mg, 6.5 mg, 7 mg, 7.5 mg, 8 mg, 8.5 mg, 9 mg, 10 mg, 11 mg, 12.5 mg, 15 mg, 17.5 mg, 20 mg, 22.5 mg, 25 mg, 27.5 mg, 30 mg, 35 mg, 40 mg, 45 mg, and 50 mg being specific examples of doses.

Typically, dosages of a compound of the present disclosure or a pharmaceutically acceptable salt thereof, are administered once, twice, three or four times daily, every other day, every three days, twice weekly, once weekly, twice monthly, once monthly, every two months, every 3 months, twice yearly, or once yearly to a patient in need thereof. In some embodiments, the dosage is about, e.g., 0.1-400 mg/administration, 0.1-300 mg/administration, 0.1-250 mg/administration, 0.1-200 mg/administration, 0.1-100 mg/administration, 0.1-50 mg/administration, or 0.1 to 25 mg/administration, for example 300 mg/administration, 250 mg/administration, 200 mg/administration, 150 mg/administration, 100 mg/administration, 75 mg/administration, 50 mg/administration, 25 mg/administration, 20 mg/administration, 10 mg/administration, 5 mg/administration, 2.5 mg/administration, 1 mg/administration, 0.5 mg/administration, 0.25 mg/administration, or 0.1 mg/administration.

In some embodiments, pharmaceutical compositions for parenteral or inhalation, e.g., a spray or mist, administration of a compound of the present disclosure or a pharmaceutically acceptable salt thereof, having a concentration of about 0.005 mg/mL to about 500 mg/mL. In some embodiments, the compositions include a compound of the present disclosure or a pharmaceutically acceptable salt thereof, at a concentration of, e.g., about 5 mg/mL to about 500 mg/mL, about 5 mg/mL to about 100 mg/mL, about 5 mg/mL to about 50 mg/mL, about 1 mg/mL to about 100 mg/mL, about 1 mg/mL to about 50 mg/mL, about 0.1 mg/mL to about 25 mg/mL, about 0.1 mg/mL to about 10 mg/mL, about 0.05 mg/mL to about 10 mg/mL, about 0.05 mg/mL to about 5 mg/mL, about 0.05 mg/mL to about 1 mg/mL, about 0.005 mg/mL to about 1 mg/mL, about 0.005 mg/mL to about 0.25 mg/mL, or about 0.005 mg/mL to about 0.1 mg/mL.

In some embodiments, the composition includes a compound of the present disclosure or a pharmaceutically acceptable salt thereof, at a concentration of, e.g., about 0.05 mg/mL to about 500 mg/mL, about 0.05 mg/mL to about 100 mg/mL, about 0.05 mg/mL to about 50 mg/mL, about 0.05 mg/mL to about 25 mg/mL, about 0.05 mg/mL to about 10 mg/mL, about 0.05 mg/mL to about 5 mg/mL, about 0.005 mg/mL to about 1 mg/mL, about 0.005 mg/mL to about 0.25 mg/mL, about 0.005 mg/mL to about 0.05 mg/mL, or about 0.005 mg/mL to about 0.025 mg/mL. In some embodiments, the pharmaceutical compositions are formulated as a total volume of about, e.g., 0.1 mL, 0.25 mL, 0.5 mL, 1 mL, 2 mL, 5 mL, 10 mL, 20 mL, 25 mL, 50 mL, 100 mL, 200 mL, 250 mL, or 500 mL.

Typically, dosages may be administered to a subject once, twice, three times or four times daily, every other day, every three days, twice weekly, once weekly, twice monthly, once monthly, thrice yearly, twice yearly, or once yearly. In some embodiments, a compound of the present disclosure or a pharmaceutically acceptable salt thereof is administered to a subject once in the morning, or once in the evening. In some embodiments, a compound of the present disclosure or a pharmaceutically acceptable salt thereof is administered to a subject once in the morning, and once in the evening. In some embodiments, a compound of the present disclosure or a pharmaceutically acceptable salt thereof is administered to a subject three times a day (e.g., at breakfast, lunch, and dinner), at a dose, e.g., of 0.5 mg/administration (e.g., 1.5 mg/day).

In some embodiments, any compound (or pharmaceutical composition thereof) disclosed in this application may be administered one, two, three, or four times per day, wherein the administrations may be the same or different and are independently selected from any of the dosages provided in this application. In some embodiments, any compound (or pharmaceutical composition thereof) disclosed in this application may be administered one, two, three, four, five, six, seven, eight, nine, or ten times per week (possibly at equally spaced or alternating intervals), wherein the administrations may be the same or different and are independently selected from any of the dosages provided in this application. In some embodiments, any compound (or pharmaceutical composition thereof) disclosed in this application may be administered one, two, three, four, five, six, seven, eight, nine, ten, eleven, twelve, thirteen, fourteen, or fifteen times per month (possibly at equally spaced or alternating intervals, such as every two, three, four, or five days, or alternating between any of these), wherein the administrations may be the same or different and are independently selected from any of the dosages provided in this application. In some embodiments, any compound (or pharmaceutical composition thereof) disclosed in this application may be administered one, two, three, four, five, six, seven, eight, nine, ten, eleven, twelve, thirteen, fourteen, fifteen, sixteen, seventeen, eighteen, nineteen, twenty, twenty-one, twenty-two, twenty-three, twenty-four, or twenty-five times per year (possibly at equally spaced or alternating intervals, such as every two, three, or four weeks, or once or twice a month, or alternating between any of these), wherein the administrations may be the same or different and are independently selected from any of the dosages provided in this application.

In some embodiments, a compound of the present disclosure or a pharmaceutically acceptable salt thereof is administered to a subject at a dose of 0.5 mg/day in one or more doses. In some embodiments, a compound of the present disclosure or a pharmaceutically acceptable salt thereof is administered to a subject at a dose of 1 mg/day in one or more doses. In some embodiments, a compound of the present disclosure or a pharmaceutically acceptable salt thereof is administered to a subject at a dose of 2.5 mg/day in one or more doses. In some embodiments, a compound of the present disclosure or a pharmaceutically acceptable salt thereof is administered to a subject at a dose of 5 mg/day in one or more doses. In some embodiments, a compound of the present disclosure or a pharmaceutically acceptable salt thereof is administered to a subject at a dose of 10 mg/day in one or more doses. In some embodiments, a compound of the present disclosure or a pharmaceutically acceptable salt thereof is administered to a subject at a dose of 15 mg/day in one or more doses. In some embodiments, a compound of the present disclosure or a pharmaceutically acceptable salt thereof is administered to a subject at a dose of 20 mg/day in one or more doses. In some embodiments, a compound of the present disclosure or a pharmaceutically acceptable salt thereof is administered to a subject at a dose of 25 mg/day in one or more doses. In some embodiments, a compound of the present disclosure or a pharmaceutically acceptable salt thereof is administered to a subject at a dose of 30 mg/day in one or more doses. In some embodiments, a compound of the present disclosure or a pharmaceutically acceptable salt thereof is administered to a subject at a dose of 40 mg/day in one or more doses. In some embodiments, a compound of the present disclosure or a pharmaceutically acceptable salt thereof is administered to a subject at a dose of 50 mg/day in one or more doses. In some embodiments, a compound of the present disclosure or a pharmaceutically acceptable salt thereof is administered to a subject at a dose of 75 mg/day in one or more doses. In some embodiments, a compound of the present disclosure or a pharmaceutically acceptable salt thereof is administered to a subject at a dose of 100 mg/day in one or more doses.

In some embodiments, the dosage of a compound of the present disclosure or a pharmaceutically acceptable salt thereof is 0.0005-5 mg/kg, 0.001-1 mg/kg, 0.01-1 mg/kg or 0.1-5 mg/kg once, twice, three times or four times daily. For example, in some embodiments, the dosage is 0.0005 mg/kg, 0.001 mg/kg, 0.005 mg/kg, 0.01 mg/kg, 0.025 mg/kg, 0.05 mg/kg, 0.1 mg/kg, 0.15 mg/kg, 0.2 mg/kg, 0.25 mg/kg, 0.3 mg/kg, 0.4 mg/kg, 0.5 mg/kg, 1 mg/kg, 2.5 mg/kg, 5 mg/kg, once, twice, three times, or four times daily. In some embodiments, a subject is administered a total daily dose of 0.01 mg to 500 mg of a compound of the present disclosure or a pharmaceutically acceptable salt thereof once, twice, three times, or four times daily. In some embodiments, the total amount administered to a subject in 24-hour period is, e.g., 0.01 mg, 0.025 mg, 0.05 mg, 0.075 mg, 0.1 mg, 0.125 mg, 0.15 mg, 0.175 mg, 0.2 mg, 0.25 mg, 0.3 mg, 0.4 mg, 0.5 mg, 0.75 mg, 1 mg, 1.5 mg, 2 mg, 2.5 mg, 3 mg, 4 mg, 5 mg, 7.5 mg, 10 mg, 12.5 mg, 15 mg, 17.5 mg 20 mg, 25 mg, 30 mg, 35 mg, 40 mg, 50 mg, 60 mg, 75 mg, 100 mg, 150 mg, 200 mg, 300 mg, 400 mg, 500 mg. In some embodiments, the subject may be started at a low dose and the dosage is escalated. In some embodiments, the subject may be started at a high dose and the dosage is decreased.

In some embodiments, a compound of the present disclosure or a pharmaceutically acceptable salt thereof is administered to a patient under the supervision of a healthcare provider.

In some embodiments, a compound of the present disclosure or a pharmaceutically acceptable salt thereof is administered to a patient under the supervision of a healthcare provider at a clinic specializing in the delivery of psychoactive treatments.

In some embodiments, a compound of the present disclosure is administered to a patient under the supervision of a healthcare provider at a high dose intended to induce a psychedelic experience in the subject, e.g., 5 mg, 7.5 mg, 10 mg, 12.5 mg, 15 mg, 17.5 mg, 20 mg, 25 mg, 30 mg, 35 mg, 40 mg, 50 mg, 60 mg, 70 mg, 80 mg, 90 mg, or 100 mg.

In some embodiments, the administration to a patient of a high dose under the supervision of a healthcare provider occurs periodically in order to maintain a therapeutic effect in the patient, e.g., every three days, twice weekly, once weekly, twice monthly, once monthly, four times yearly, thrice yearly, twice yearly, or once yearly.

In some embodiments, a compound of the present disclosure or a pharmaceutically acceptable salt thereof is administered by a patient on their own at home or otherwise away from the supervision of a healthcare provider.

In some embodiments, a compound of the present disclosure or a pharmaceutically acceptable salt thereof is administered by a patient on their own at home or otherwise away from the supervision of a healthcare provider at a low dose intended to be sub-perceptual or to induce threshold psychoactive effects, e.g., 0.1 mg, 0.25 mg, 0.5 mg, 0.75 mg, 1 mg, 1.5 mg, 2 mg, 2.5 mg, 3 mg, or 4 mg.

In some embodiments, the administration by a patient of a low dose on their own occurs periodically in order to maintain a therapeutic effect in the patient, e.g., daily, every other day, every three days, twice weekly, once weekly, twice monthly, or once monthly,

In some embodiments, a compound of the present disclosure or a pharmaceutically acceptable salt thereof may be administered, e.g., via inhalation or orally, at specified intervals. For example, during treatment a patient may be administered a compound of the present disclosure at intervals of every, e.g., 1 year, 6 months, 4 months, 90 days, 60 days, 30 days, 14 days, 7 days, 3 days, 24 hours, 12 hours, 8 hours, 6 hours, 5 hours, 4 hours, 3 hours, 2.5 hours, 2.25 hours, 2 hours, 1.75 hours, 1.5 hours, 1.25 hours, 1 hour, 0.75 hour, 0.5 hour, or 0.25 hour.

### EXAMPLES

The compounds described herein can be prepared in a number of ways based on the teachings contained herein and synthetic procedures well known in the art. In the description of the synthetic methods described below, it is to be understood that all proposed reaction conditions, including choice of solvent, reaction atmosphere, reaction temperature, duration of the experiment, and workup procedures, can be chosen to be the conditions standard for that reaction, unless otherwise indicated. It is understood by one skilled in the art of organic synthesis that the functionality present on various portions of the molecule should be compatible with the reagents and reactions proposed. Substituents not compatible with the reaction conditions will be apparent to one skilled in the art, and alternate methods are therefore indicated. The starting materials for the examples are either commercially available or are readily prepared by standard methods from known materials.

At least some of the compounds identified as “Intermediates” herein are contemplated as compounds of the disclosure.
DPPA=diphenylphosphoryl azide
TEA=triethylamine
BnOH=n-benzyl alcohol
Cbz=benzyloxycarbonyl
NBS=N-bromosuccinimide
MeCN=acetonitrile
DIPEA=N,N-diisopropylethylamine
DTBBPY=4,4′-di-tert-butyl-2,2′-bipyridine
TTMSS=tris(trimethylsilyl)silane
DPPF=1,1′-bis(diphenylphosphino)ferrocene
DME=dimethoxyethane
DCM=dichloromethane

### General Procedures

The compounds of the present disclosure may be prepared by techniques well known in organic synthesis and familiar to a practitioner ordinarily skilled in the art. For example, the compounds may be prepared by the chemical transformations described in the following schemes and examples. However, these may not be the only means by which to synthesize or obtain the desired compounds.

The intermediate benzyl (1-(4-bromo-2,5-dimethoxyphenyl)butan-2-yl)carbamate was generated as depicted in Scheme 1 using a Curtis rearrangement as the key step. In Scheme 2, a Henry reaction followed by reduction of the nitro group was used to make benzyl (1-(4-bromo-2,5-dimethoxyphenyl)butan-2-yl)carbamate.

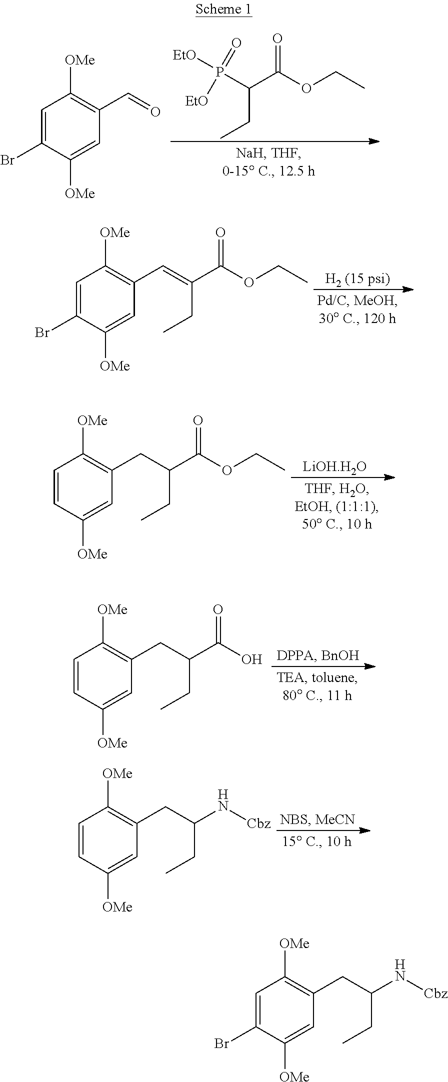

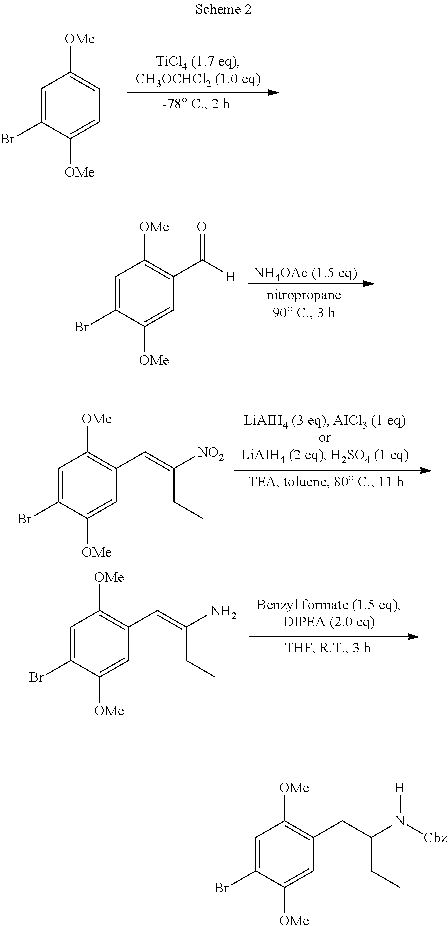

Compounds 2a, 2b, and 3a can be prepared from benzyl (1-(4-bromo-2,5-dimethoxyphenyl)butan-2-yl)carbamate as depicted in Scheme 3, Scheme 4, or Scheme 5. In Scheme 3, a photosensitized nickel-catalyzed coupling is used in the key step. In Scheme 4, an acetylenic coupling followed by reduction are used as the key steps. In Scheme 5, a palladium-catalyzed boronic acid coupling is used as the key step.

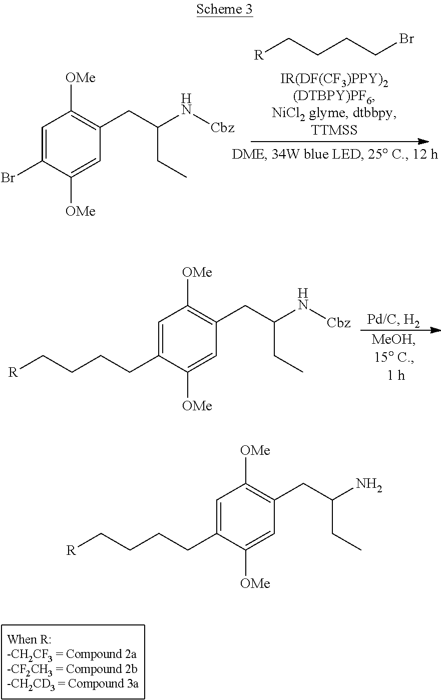

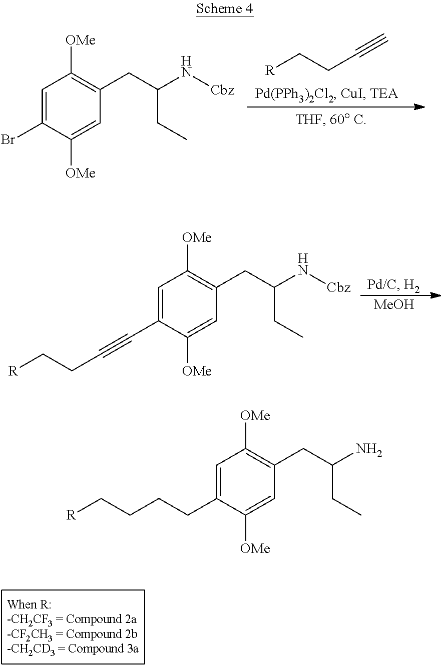

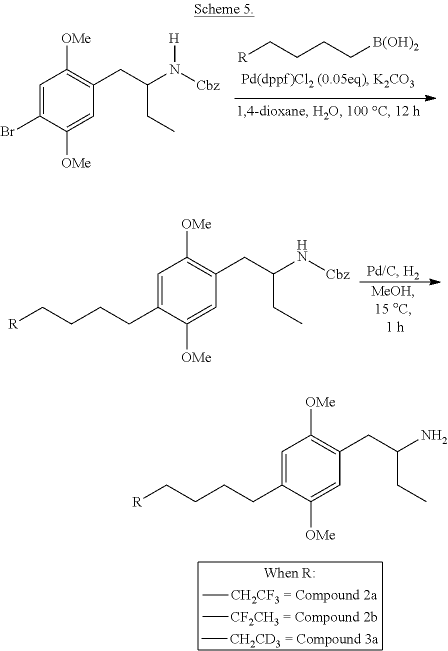

Alternative approaches to Compound 2b are depicted in Scheme 6 and Scheme 7.

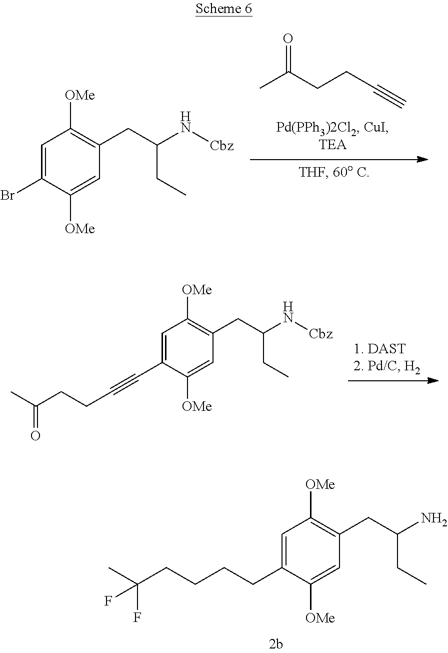

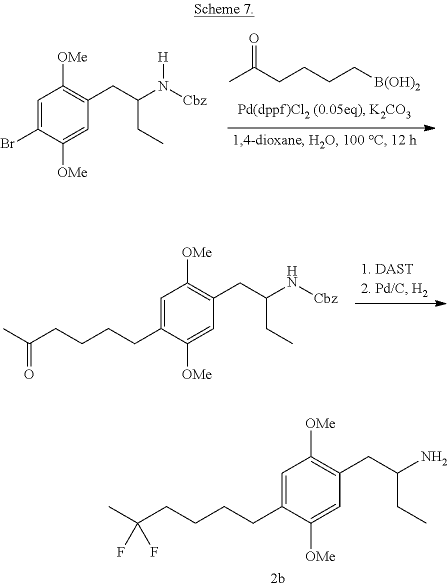

### Example 1. Preparation of (R)-1-(4-hexyl-2,5-dimethoxyphenyl)butan-2-amine (1R) and (S)-1-(4-hexyl-2,5-dimethoxyphenyl)butan-2-amine (1S)

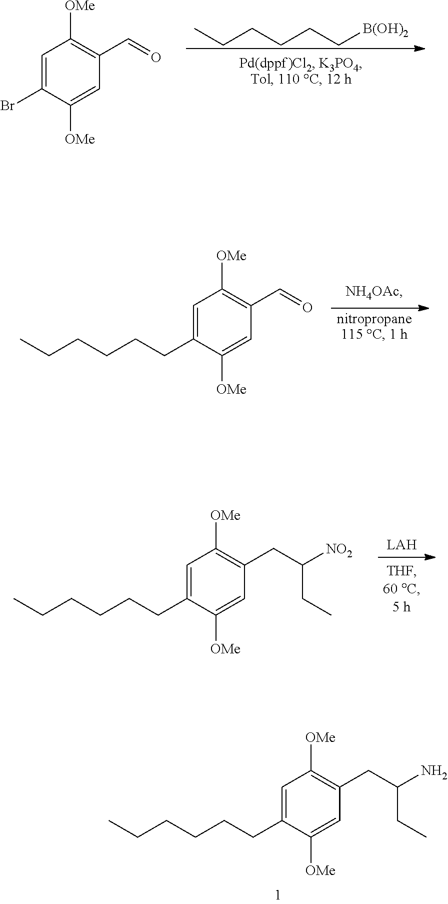

### Step 1: Preparation of 4-hexyl-2,5-dimethoxybenzaldehyde

4-bromo-2,5-dimethoxybenzaldehyde (5 g, 20.40 mmol, 1 eq), hexylboronic acid (2.65 g, 20.40 mmol, 1 eq), Pd(dppf)Cl₂ (746.42 mg, 1.02 mmol, 0.05 eq), and K₃PO₄ (8.66 g, 40.80 mmol, 2 eq) in toluene (50 mL) was de-gassed and then heated to 110° C. for 12 h under N₂. On completion, the mixture was filtered and concentrated. The residue was purified by silica gel chromatography eluted with petroleum ether/ethyl acetate gradient (100:1 to 50:1) to give 4-hexyl-2,5-dimethoxybenzaldehyde (4.2 g, 16.78 mmol, 82.23% yield) as a yellow solid. ¹H NMR (400 MHz, CHLOROFORM-d) δ ppm=10.40 (s, 1H), 7.27 (s, 1H), 6.80 (s, 1H), 3.90 (s, 3H), 3.83 (s, 3H), 2.69-2.60 (m, 2H), 1.67-1.53 (m, 2H), 1.42-1.27 (m, 6H), 0.94-0.85 (m, 3H).

### Step 2: Preparation of (E)-1-hexyl-2,5-dimethoxy-4-(2-nitrobut-1-en-1-yl)benzene

A mixture of 4-hexyl-2,5-dimethoxy-benzaldehyde (1.8 g, 7.19 mmol, 1 eq) and NH₄OAc (1.11 g, 14.38 mmol, 2 eq) in 1-nitropropane (24.92 g, 279.75 mmol, 24.97 mL, 38.91 eq) was heated at 115° C. for 1 h. On completion, the mixture was concentrated. The residue was purified by silica gel chromatography eluted with petroleum ether/ethyl acetate (30:1) to give (E)-1-hexyl-2,5-dimethoxy-4-(2-nitrobut-1-en-1-yl)benzene (1 g, 3.11 mmol, 43.27% yield) as a yellow solid. ¹H NMR (400 MHz, CHLOROFORM-d) 8 ppm=8.26 (s, 1H), 6.78 (d, J=15.6 Hz, 2H), 3.83 (dd, J=1.2, 14.4 Hz, 6H), 2.87 (m, 2H), 2.69-2.58 (m, 1H), 2.69-2.58 (m, 1H), 1.67-1.51 (m, 3H), 1.45-1.25 (m, 11H), 0.97-0.85 (m, 3H).

### Step 3: Preparation of 1-(4-hexyl-2,5-dimethoxyphenyl)butan-2-amine (1) hydrochloride

A solution of (E)-1-hexyl-2,5-dimethoxy-4-(2-nitrobut-1-en-1-yl)benzene (1 g, 3.11 mmol, 1 eq) in THF (10 mL) was cooled to 0° C. Then LAH (472.29 mg, 12.45 mmol, 4 eq) was added. The mixture was warmed to 60° C. and stirred at 60° C. for 5 h. On completion, the mixture was cooled to 0° C. Then 0.5 mL H₂O was added followed by 0.5 mL 30% aq. NaOH. The mixture was filtered and concentrated. The residue was purified by prep-HPLC (Column: Phenomenex Luna C18 250×50 mm×10 μm; [mobile phase: water (0.04% HCl)-ACN]; B %: 20%-50%, 10 min) to afford 1-(4-hexyl-2,5-dimethoxyphenyl)butan-2-amine hydrochloride (1 HCl, 380 mg, 1.15 mmol, 37.02% yield, 100% purity) as a white solid. ¹H NMR (400 MHz, DMSO-d₆) δ ppm=7.96 (br s, 3H), 6.80 (d, J=10.8 Hz, 2H), 3.73 (s, 6H), 3.33 (s, 12H), 3.28-3.18 (m, 1H), 2.79 (d, J=6.8 Hz, 2H), 2.53 (s, 1H), 2.56-2.52 (m, 1H), 1.57-1.44 (m, 4H), 1.28 (s, 6H), 0.96-0.81 (m, 6H). ¹³C NMR (101 MHz, DMSO-d₆) δ ppm=151.01, 150.71, 129.78, 122.16, 114.05, 113.01, 55.85, 52.22, 32.64, 31.12, 29.67, 29.57, 28.66, 24.78, 22.08, 13.97, 9.45.

### Step 4: Chiral SFC separation of (R)-1-(4-hexyl-2,5-dimethoxyphenyl)butan-2-amine (1R) and (S)-1-(4-hexyl-2,5-dimethoxyphenyl)butan-2-amine (1S)

The racemate (1) was separated by chiral SFC (Instrument: Waters SFC350 preparative SFC; Column: DAICEL CHIRALPAK IC (250 mm*50 mm, 10 μm); Mobile Phase: A: CO₂, B: 0.1% NH₃H₂O in MeOH, B %: 30% isocratic; Flow rate: 200 g/min; Wavelength: 220 nm; Column Temperature: 40° C.; System Back Pressure: 100 bar; multi-injection process) to provide ENT-1 2.458 min (1R) and ENT-2 2.796 min (1S). Retention times were determined using a separate chiral analytical method.

### Example 2. X-Ray Crystal Structure of (R)-1-(4-hexyl-2,5-dimethoxyphenyl)butan-2-amine (1R) hydrochloride

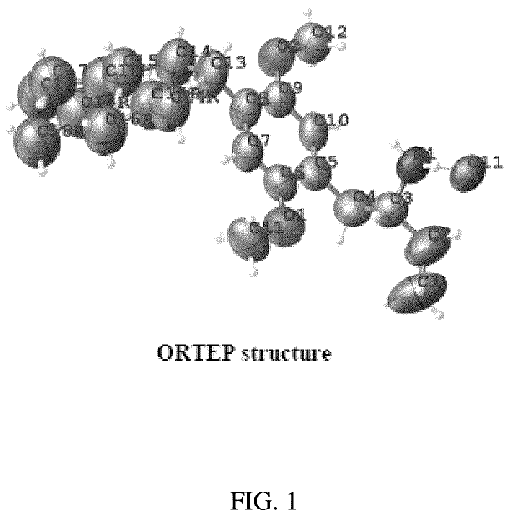

Summary of Results. A single crystal of the hydrochloride salt of ENT-1 2.458 min was grown and the structure assigned by x-ray crystallography. This isomer was found to correspond to the R isomer (1R). The crystal was a colorless needle with the following dimensions: 0.30×0.04×0.02 mm³. The symmetry of the crystal structure was assigned the monoclinic space group P21 with the following parameters: a=16.390 (2) Å, b=5.4663 (3) Å, c=23.706 (2) Å, α=90°, β=106.162(12)°, γ=90°, V=2039.9(4) Å3, Z=4, Dc=1.074 g/cm3, F(000)=720.0, μ(CuKα)=1.698 mm-1, and T=293 (2) K. The ORTEP structure is shown in FIG. 1.

Rigaku Oxford Diffraction XtaLAB Synergy-S four-circle diffractometer equipped with a HyPix-6000HE area detector.
Cryogenic system: Oxford Cryostream 800
Cu: λ=1.54184 Å, 50W, Micro focus source with multilayer mirror (μ-CMF).
Distance from the crystal to the CCD detector: d=35 mm
Tube Voltage: 50 kV
Tube Current: 1 mA

A total of 35407 reflections were collected in the 2θ range from 5.614 to 133.17. The limiting indices were: −19≤h≤19, −6≤k≤6, −24≤1≤28; which yielded 6985 unique reflections (Rint=0.1064). The structure was solved using SHELXT (Sheldrick, G. M. 2015. Acta Cryst. A71, 3-8) and refined using SHELXL (against F²) (Sheldrick, G. M. 2015. Acta Cryst. C71, 3-8). The total number of refined parameters was 528, compared with 6985 data. All reflections were included in the refinement. The goodness of fit on F² was 1.049 with a final R value for [I>2σ (I)] R1=0.0870 and wR2=0.2425. The largest differential peak and hole were 0.28 and −0.18 Å-3, respectively.

Description of Crystal Preparation. 14 mg of 1R HCl (ENT-1 12.458 min HCl) was dissolved in 1.6 mL acetonitrile/methanol (3:1) and kept in a half-sealed 4 mL vial. The solution was evaporated slowly at 50° C. Crystals were observed during the second day.

### X-Ray Data Tables.

TABLE 1
Summary of X-ray crystallographic data.

| Crystal size | 0.30 × 0.04 × 0.02 mm³ | |
|---|---|---|
| Radiation Type | CuKα (λ = 1.54184) | |
| Crystal system | monoclinic | |
| Space group | P21 | |
| a/Å | 16.390 | (2) |
| b/Å | 5.4663 | (3) |
| c/Å | 23.706 | (2) |
| α/° | 90 | |
| β/° | 106.162 | (12) |
| γ/° | 90 | |
| Cell Volume/Å3 | 2039.9 | (4) |
| Cell Formula Units Z | 4 | |
| Crystal Density calc g/cm3 | 1.074 | |
| Crystal F(000) | 720.0 | |
| Absorption Coefficient μ/ | 1.698 | |
| mm − 1 | | |
| Index ranges | −19 ≤ h ≤ 19, −6 ≤ k ≤ 6, −24 ≤ | |
| | l ≤ 28 | |
| Cell Measurement Temperature/K | 293 | (2) |
| 2θ range for data collection/° | 5.614 to 133.17 | |
| Goodness-of-fit on F2 | 1.049 | |
| Final R indexes [I >= 2σ (I)] | R1 = 0.0870, wR2 = 0.2425 | |
| Final R indexes [all data] | R1 = 0.1274, wR2 = 0.2756 | |
| Largest diff. peak/hole/e Å − 3 | 0.28/−0.18 | |
| Reflections collected/unique | 35407/6985 [Rint = 0.1064] | |
| Flack parameter | 0.04 | (2) |

TABLE 2
Atomic coordinates (×10\hat{ }4) and equivalent isotropic displacement
parameters (A\hat{ }2 × 10\hat{ }3).

| Atom | x | | y | | z | | U(eq) | |
|---|---|---|---|---|---|---|---|---|
| Cl(1) | 5247.9 | (15) | 883 | (4) | 5743.0 | (8) | 106.1 | (7) |
| Cl(1A) | 6097.2 | (14) | 8744 | (4) | 254.3 | (9) | 105.1 | (7) |
| O(2) | 6625 | (5) | 10297 | (12) | 3654 | (3) | 123 | (2) |
| N(1A) | 5657 | (4) | 3775 | (13) | 754 | (3) | 93.6 | (16) |
| O(1A) | 5071 | (5) | 5337 | (14) | 2335 | (3) | 126 | (2) |
| N(1) | 6032 | (5) | 5848 | (13) | 5533 | (3) | 102.1 | (18) |
| O(1) | 8505 | (5) | 4168 | (16) | 5390 | (3) | 139 | (2) |
| O(2A) | 2622 | (6) | −254 | (16) | 818 | (4) | 157 | (3) |
| C(5A) | 4720 | (7) | 2239 | (13) | 1634 | (3) | 102 | (3) |
| C(10A) | 4109 | (8) | 836 | (17) | 1253 | (4) | 111 | (3) |
| C(5) | 7515 | (6) | 7416 | (16) | 5098 | (4) | 101 | (2) |
| C(6) | 8045 | (6) | 5658 | (17) | 4938 | (4) | 108 | (2) |
| C(6A) | 4448 | (7) | 3979 | (17) | 1976 | (3) | 108 | (3) |
| C(9) | 7081 | (6) | 8832 | (17) | 4094 | (4) | 103 | (2) |
| C(9A) | 3265 | (9) | 1046 | (19) | 1201 | (4) | 121 | (3) |
| C(8A) | 3004 | (9) | 2750 | (20) | 1563 | (6) | 135 | (3) |
| C(7A) | 3595 | (8) | 4182 | (18) | 1936 | (4) | 122 | (3) |
| C(3A) | 6034 | (5) | 3910 | (20) | 1402 | (4) | 116 | (3) |
| C(4A) | 5654 | (7) | 1905 | (18) | 1697 | (4) | 118 | (3) |
| C(7) | 8080 | (6) | 5580 | (20) | 4375 | (4) | 114 | (3) |
| C(10) | 7046 | (5) | 8933 | (16) | 4677 | (4) | 98 | (2) |
| C(8) | 7603 | (7) | 7070 | (17) | 3935 | (5) | 116 | (3) |
| C(4) | 7457 | (7) | 7510 | (20) | 5734 | (4) | 123 | (3) |
| C(3) | 6923 | (7) | 5587 | (19) | 5895 | (3) | 115 | (2) |
| C(12) | 6025 | (8) | 11950 | (19) | 3773 | (5) | 128 | (3) |
| C(12A) | 2891 | (11) | −1910 | (20) | 431 | (6) | 165 | (5) |
| C(11A) | 4819 | (9) | 7140 | (20) | 2691 | (4) | 134 | (4) |
| C(13A) | 2082 | (10) | 2870 | (30) | 1551 | (8) | 184 | (5) |
| C(13) | 7603 | (9) | 6820 | (20) | 3313 | (6) | 159 | (4) |
| C(11) | 9085 | (8) | 2410 | (30) | 5261 | (7) | 155 | (4) |
| C(15A) | 639 | (16) | 5110 | (60) | 1164 | (18) | 198 | (5) |
| C(14A) | 1567 | (15) | 4780 | (60) | 1147 | (17) | 187 | (6) |
| C(16A) | 140 | (30) | 6710 | (110) | 650 | (30) | 214 | (7) |
| C(2A) | 6970 | (10) | 4490 | (50) | 1470 | (20) | 150 | (7) |
| C(2) | 6871 | (10) | 5650 | (40) | 6533 | (4) | 185 | (5) |
| C(1) | 7553 | (14) | 4070 | (50) | 6936 | (7) | 251 | (10) |
| C(1A) | 7457 | (13) | 2100 | (60) | 1474 | (19) | 172 | (9) |
| C(2B) | 6963 | (8) | 3050 | (40) | 1608 | (13) | 147 | (6) |
| C(1B) | 7525 | (17) | 4980 | (50) | 1437 | (14) | 173 | (8) |
| C(17B) | −630 | (20) | 7270 | (80) | 1007 | (19) | 223 | (7) |
| C(18A) | −1160 | (20) | 8900 | (70) | 754 | (16) | 253 | (10) |
| C(14B) | 1710 | (30) | 5450 | (50) | 1510 | (40) | 189 | (6) |
| C(15B) | 760 | (30) | 5180 | (140) | 1460 | (40) | 195 | (6) |
| C(16B) | 310 | (20) | 7130 | (80) | 1010 | (20) | 211 | (6) |
| C(18B) | −910 | (50) | 8310 | (190) | 1530 | (30) | 246 | (13) |
| C(17A) | −830 | (30) | 6530 | (90) | 540 | (30) | 222 | (7) |
| C(15R) | 8862 | (12) | 9250 | (40) | 3418 | (10) | 187 | (5) |
| C(14) | 8000 | (13) | 8970 | (30) | 3063 | (9) | 178 | (5) |
| C(16) | 9240 | (15) | 11120 | (50) | 2959 | (11) | 210 | (5) |
| C(17R) | 9999 | (17) | 11800 | (50) | 3207 | (16) | 216 | (6) |
| C(18) | 10362 | (14) | 13650 | (50) | 2836 | (11) | 231 | (8) |
| C(14R) | 8576 | (12) | 6600 | (50) | 3450 | (40) | 181 | (5) |
| C(15) | 8800 | (40) | 8610 | (90) | 2850 | (50) | 191 | (5) |
| C(17) | 9950 | (30) | 11150 | (80) | 2650 | (30) | 220 | (7) |
| C(16R) | 9808 | (17) | 9300 | (80) | 3440 | (50) | 202 | (6) |
| C(18R) | 10960 | (30) | 12000 | (200) | 3290 | (110) | 222 | (11) |

TABLE 3
Bond lengths [A].

| Atom | Atom | Length/Å | Atom | Atom | Length/Å |
|---|---|---|---|---|---|
| O(2) | C(9) | 1.362(11) | C(7) | C(8) | 1.383(14) |
| O(2) | C(12) | 1.420(11) | C(8) | C(13) | 1.481(16) |
| N(1A) | C(3A) | 1.489(11) | C(4) | C(3) | 1.486(14) |
| O(1A) | C(6A) | 1.353(12) | C(3) | C(2) | 1.539(10) |
| O(1A) | C(11A) | 1.431(12) | C(13A) | C(14A) | 1.507(13) |
| N(1) | C(3) | 1.480(11) | C(13A) | C(14B) | 1.530(14) |
| O(1) | C(6) | 1.390(12) | C(13) | C(14) | 1.535(11) |
| O(1) | C(11) | 1.443(14) | C(13) | C(14R) | 1.541(12) |
| O(2A) | C(9A) | 1.382(14) | C(15A) | C(14A) | 1.545(13) |
| O(2A) | C(12A) | 1.442(15) | C(15A) | C(16A) | 1.537(14) |
| C(5A) | C(10A) | 1.379(13) | C(16A) | C(17A) | 1.541(14) |
| C(5A) | C(6A) | 1.402(12) | C(2A) | C(1A) | 1.530(12) |
| C(5A) | C(4A) | 1.507(14) | C(2) | C(1) | 1.520(12) |
| C(10A) | C(9A) | 1.361(14) | C(2B) | C(1B) | 1.529(12) |
| C(5) | C(6) | 1.417(13) | C(17B) | C(16B) | 1.539(14) |
| C(5) | C(10) | 1.359(13) | C(17B) | C(18B) | 1.539(14) |
| C(5) | C(4) | 1.538(14) | C(18A) | C(17A) | 1.540(14) |
| C(6) | C(7) | 1.350(13) | C(14B) | C(15B) | 1.538(14) |
| C(6A) | C(7A) | 1.379(14) | C(15B) | C(16B) | 1.539(14) |
| C(9) | C(10) | 1.399(12) | C(15R) | C(14R) | 1.533(12) |
| C(9) | C(8) | 1.408(12) | C(15R) | C(16R) | 1.536(12) |
| C(9A) | C(8A) | 1.409(15) | C(14) | C(15) | 1.541(12) |
| C(8A) | C(7A) | 1.363(16) | C(16) | C(15) | 1.539(12) |
| C(8A) | C(13A) | 1.50(2) | C(16) | C(17) | 1.536(12) |
| C(3A) | C(4A) | 1.523(15) | C(17R) | C(16R) | 1.543(12) |
| C(3A) | C(2A) | 1.531(12) | C(17R) | C(18R) | 1.54(2) |
| C(3A) | C(2B) | 1.538(11) | C(18) | C(17) | 1.539(12) |

TABLE 4
Bond angles |deg|.

| Atom | Atom | Atom | Length/Å | Atom | Atom | Atom | Length/Å |
|---|---|---|---|---|---|---|---|
| C(9) | O(2) | C(12) | 119.0(7) | C(6) | C(7) | C(8) | 123.7(9) |
| C(6A) | O(1A) | C(11A) | 117.1(9) | C(5) | C(10) | C(9) | 122.1(8) |
| C(6) | O(1) | C(11) | 118.3(9) | C(9) | C(8) | C(13) | 119.8(9) |
| C(9A) | O(2A) | C(12A) | 115.4(10) | C(7) | C(8) | C(9) | 117.3(8) |
| C(10A) | C(5A) | C(6A) | 117.8(10) | C(7) | C(8) | C(13) | 122.8(9) |
| C(10A) | C(5A) | C(4A) | 122.0(8) | C(3) | C(4) | C(5) | 115.4(7) |
| C(6A) | C(5A) | C(4A) | 120.1(9) | N(1) | C(3) | C(4) | 109.9(8) |
| C(9A) | C(10A) | C(5A) | 123.2(9) | N(1) | C(3) | C(2) | 104.7(8) |
| C(6) | C(5) | C(4) | 119.7(9) | C(4) | C(3) | C(2) | 115.8(11) |
| C(10) | C(5) | C(6) | 118.7(8) | C(8A) | C(13A) | C(14A) | 115.0(14) |
| C(10) | C(5) | C(4) | 121.5(9) | C(8A) | C(13A) | C(14B) | 115(2) |
| O(1) | C(6) | C(5) | 115.6(8) | C(8) | C(13) | C(14) | 115.6(12) |
| C(7) | C(6) | O(1) | 125.4(8) | C(8) | C(13) | C(14R) | 94(3) |
| C(7) | C(6) | C(5) | 119.0(9) | C(16A) | C(15A) | C(14A) | 111(3) |
| O(1A) | C(6A) | C(5A) | 115.5(9) | C(13A) | C(14A) | C(15A) | 116(2) |
| O(1A) | C(6A) | C(7A) | 124.9(8) | C(15A) | C(16A) | C(17A) | 113(4) |
| C(7A) | C(6A) | C(5A) | 119.6(9) | C(1A) | C(2A) | C(3A) | 109.2(12) |
| O(2) | C(9) | C(10) | 124.7(8) | C(1) | C(2) | C(3) | 111.9(12) |
| O(2) | C(9) | C(8) | 116.2(8) | C(1B) | C(2B) | C(3A) | 108.8(16) |
| C(10) | C(9) | C(8) | 119.1(8) | C(16B) | C(17B) | C(18B) | 122(5) |
| O(2A) | C(9A) | C(8A) | 115.8(12) | C(13A) | C(14B) | C(15B) | 107(4) |
| C(10A) | C(9A) | O(2A) | 125.8(10) | C(14B) | C(15B) | C(16B) | 105(4) |
| C(10A) | C(9A) | C(8A) | 118.3(11) | C(17B) | C(16B) | C(15B) | 109(3) |
| C(9A) | C(8A) | C(13A) | 119.3(13) | C(18A) | C(17A) | C(16A) | 109(4) |
| C(7A) | C(8A) | C(9A) | 119.6(11) | C(14R) | C(15R) | C(16R) | 109.6(12) |
| C(7A) | C(8A) | C(13A) | 121.1(11) | C(13) | C(14) | C(15) | 121(3) |
| C(8A) | C(7A) | C(6A) | 121.5(9) | C(17) | C(16) | C(15) | 108(2) |
| N(1A) | C(3A) | C(4A) | 109.5(7) | C(16R) | C(17R) | C(18R) | 108.1(14) |
| N(1A) | C(3A) | C(2A) | 103.4(19) | C(15R) | C(14R) | C(13) | 103.0(16) |
| N(1A) | C(3A) | C(2B) | 113.8(13) | C(16) | C(15) | C(14) | 103.6(19) |
| C(4A) | C(3A) | C(2A) | 129.0(16) | C(16) | C(17) | C(18) | 102(2) |
| C(4A) | C(3A) | C(2B) | 97.5(11) | C(15R) | C(16R) | C(17R) | 107.8(12) |

| | | | |
|---|---|---|---|
| C(5A) | C(4A) | C(3A) | 113.6(7) |

TABLE 5
Torsion angles [deg].

| A | B | C | D | Angle/° | A | B | C | D | Angle/° |
|---|---|---|---|---|---|---|---|---|---|
| O(2) | C(9) | C(10) | C(5) | 179.8(8) | C(4A) | C(3A) | C(2A) | C(1A) | 42(4) |
| O(2) | C(9) | C(8) | C(7) | 179.3(9) | C(4A) | C(3A) | C(2B) | C(1B) | −172(2) |
| O(2) | C(9) | C(8) | C(13) | −2.6(15) | C(7) | C(8) | C(13) | C(14) | −113.0(14) |
| N(1A) | C(3A) | C(4A) | C(5A) | −66.1(9) | C(7) | C(8) | C(13) | C(14R) | −45.5(16) |
| N(1A) | C(3A) | C(2A) | C(1A) | −87(3) | C(10) | C(5) | C(6) | O(1) | −179.9(8) |
| N(1A) | C(3A) | C(2B) | C(1B) | 72(2) | C(10) | C(5) | C(6) | C(7) | 1.1(13) |
| O(1A) | C(6A) | C(7A) | C(8A) | 179.9(9) | C(10) | C(5) | C(4) | C(3) | 100.8(11) |
| N(1) | C(3) | C(2) | C(1) | 147.7(15) | C(10) | C(9) | C(8) | C(7) | −2.2(14) |
| O(1) | C(6) | C(7) | C(8) | 179.1(9) | C(10) | C(9) | C(8) | C(13) | 175.8(10) |
| O(2A) | C(9A) | C(8A) | C(7A) | −177.2(10) | C(8) | C(9) | C(10) | C(5) | 1.5(13) |
| O(2A) | C(9A) | C(8A) | C(13A) | 5.7(16) | C(8) | C(13) | C(14) | C(15) | 117(5) |
| C(5A) | C(10A) | C(9A) | O(2A) | 178.1(8) | C(8) | C(13) | C(14R) | C(15R) | −92(4) |
| C(5A) | C(10A) | C(9A) | C(8A) | −1.2(14) | C(4) | C(5) | C(6) | O(1) | −1.9(12) |
| C(5A) | C(6A) | C(7A) | C(8A) | −1.0(13) | C(4) | C(5) | C(6) | C(7) | 179.1(9) |
| C(10A) | C(5A) | C(6A) | O(1A) | −178.9(7) | C(4) | C(5) | C(10) | C(9) | −178.9(8) |
| C(10A) | C(5A) | C(6A) | C(7A) | 1.9(11) | C(4) | C(3) | C(2) | C(1) | −91.2(17) |
| C(10A) | C(5A) | C(4A) | C(3A) | 105.0(9) | C(12) | O(2) | C(9) | C(10) | −4.4(13) |
| C(10A) | C(9A) | C(8A) | C(7A) | 2.2(15) | C(12) | O(2) | C(9) | C(8) | 174.0(9) |
| C(10A) | C(9A) | C(8A) | C(13A) | −174.9(11) | C(12A) | O(2A) | C(9A) | C(10A) | −2.0(15) |
| C(5) | C(6) | C(7) | C(8) | −2.1(16) | C(12A) | O(2A) | C(9A) | C(8A) | 177.3(10) |
| C(5) | C(4) | C(3) | N(1) | −62.6(11) | C(11A) | O(1A) | C(6A) | C(5A) | 179.9(7) |
| C(5) | C(4) | C(3) | C(2) | 179.2(10) | C(11A) | O(1A) | C(6A) | C(7A) | −0.9(12) |
| C(6) | C(5) | C(10) | C(9) | −0.9(13) | C(13A) | C(8A) | C(7A) | C(6A) | 175.9(11) |
| C(6) | C(5) | C(4) | C(3) | −77.2(12) | C(13A) | C(14B) | C(15B) | C(16B) | −140(6) |
| C(6) | C(7) | C(8) | C(9) | 2.6(16) | C(13) | C(14) | C(15) | C(16) | −150(4) |
| C(6) | C(7) | C(8) | C(13) | −175.3(11) | C(11) | O(1) | C(6) | C(5) | −177.4(9) |
| C(6A) | C(5A) | C(10A) | C(9A) | −0.8(12) | C(11) | O(1) | C(6) | C(7) | 1.5(15) |
| C(6A) | C(5A) | C(4A) | C(3A) | −76.4(10) | C(15A) | C(16A) | C(17A) | C(18A) | 107(6) |
| C(9) | C(8) | C(13) | C(14) | 69.1(17) | C(14A) | C(15A) | C(16A) | C(17A) | 164(4) |
| C(9) | C(8) | C(13) | C(14R) | 136.6(12) | C(16A) | C(15A) | C(14A) | C(13A) | −169(4) |
| C(9A) | C(8A) | C(7A) | C(6A) | −1.1(16) | C(2A) | C(3A) | C(4A) | C(5A) | 166.9(19) |
| C(9A) | C(8A) | C(13A) | C(14A) | −95(2) | C(2B) | C(3A) | C(4A) | C(5A) | 175.4(13) |
| C(9A) | C(8A) | C(13A) | C(14B) | −134(4) | C(14B) | C(15B) | C(16B) | C(17B) | −171(6) |
| C(8A) | C(13A) | C(14A) | C(15A) | −175(3) | C(18B) | C(17B) | C(16B) | C(15B) | 69(8) |
| C(8A) | C(13A) | C(14B) | C(15B) | 176(5) | C(14R) | C(15R) | C(16R) | C(17R) | 162(6) |
| C(7A) | C(8A) | C(13A) | C(14A) | 88(3) | C(15) | C(16) | C(17) | C(18) | −176(6) |
| C(7A) | C(8A) | C(13A) | C(14B) | 49(4) | C(17) | C(16) | C(15) | C(14) | −169(6) |
| C(4A) | C(5A) | C(10A) | C(9A) | 177.8(8) | C(16R) | C(15R) | C(14R) | C(13) | −170(6) |
| C(4A) | C(5A) | C(6A) | O(1A) | 2.5(10) | C(18R) | C(17R) | C(16R) | C(15R) | 174(11) |

| | | | | |
|---|---|---|---|---|
| C(4A) | C(5A) | C(6A) | C(7A) | −176.7(8) |

TABLE 6
Atomic occupancy.

| Atom | Occupancy | Atom | Occupancy | Atom | Occupancy |
|---|---|---|---|---|---|
| H(3A) | 0.42(3) | H(3AA) | 0.58(3) | H(13A) | 0.63(4) |
| H(13B) | 0.63(4) | H(13C) | 0.37(4) | H(13D) | 0.37(4) |
| H(13E) | 0.80(2) | H(13F) | 0.80(2) | H(13G) | 0.20(2) |
| H(13H) | 0.20(2) | C(15A) | 0.71(7) | H(15A) | 0.71(7) |
| H(15B) | 0.71(7) | C(14A) | 0.63(4) | H(14A) | 0.63(4) |
| H(14B) | 0.63(4) | C(16A) | 0.44(5) | H(16A) | 0.44(5) |
| H(16B) | 0.44(5) | C(2A) | 0.42(3) | H(2AA) | 0.42(3) |
| H(2AB) | 0.42(3) | C(1A) | 0.43(3) | H(1AD) | 0.43(3) |
| H(1AE) | 0.43(3) | H(1AF) | 0.43(3) | C(2B) | 0.58(3) |
| H(2BA) | 0.58(3) | H(2BB) | 0.58(3) | C(1B) | 0.57(3) |
| H(1BA) | 0.57(3) | H(1BB) | 0.57(3) | H(1BC) | 0.57(3) |
| C(17B) | 0.62(3) | H(17A) | 0.62(3) | H(17B) | 0.62(3) |
| C(18A) | 0.74(3) | H(18A) | 0.74(3) | H(18B) | 0.74(3) |
| H(18C) | 0.74(3) | C(14B) | 0.37(4) | H(14C) | 0.37(4) |
| H(14D) | 0.37(4) | C(15B) | 0.29(7) | H(15C) | 0.29(7) |
| H(15D) | 0.29(7) | C(16B) | 0.56(5) | H(16C) | 0.56(5) |
| H(16D) | 0.56(5) | C(18B) | 0.26(3) | H(18D) | 0.26(3) |
| H(18E) | 0.26(3) | H(18F) | 0.26(3) | C(17A) | 0.38(3) |
| H(17C) | 0.38(3) | H(17D) | 0.38(3) | C(15R) | 0.86(2) |
| H(15E) | 0.86(2) | H(15F) | 0.86(2) | C(14) | 0.80(2) |
| H(14E) | 0.80(2) | H(14F) | 0.80(2) | C(16) | 0.87(3) |
| H(16E) | 0.87(3) | H(16F) | 0.87(3) | C(17R) | 0.67(3) |
| H(17E) | 0.67(3) | H(17F) | 0.67(3) | C(18) | 0.91(2) |
| H(18G) | 0.91(2) | H(18H) | 0.91(2) | H(18I) | 0.91(2) |
| C(14R) | 0.20(2) | H(14G) | 0.20(2) | H(14H) | 0.20(2) |
| C(15) | 0.14(2) | H(15G) | 0.14(2) | H(15H) | 0.14(2) |
| C(17) | 0.33(3) | H(17G) | 0.33(3) | H(17H) | 0.33(3) |
| C(16R) | 0.13(3) | H(16G) | 0.13(3) | H(16H) | 0.13(3) |
| C(18R) | 0.09(2) | H(18J) | 0.09(2) | H(18K) | 0.09(2) |

| | |
|---|---|
| H(18L) | 0.09(2) |

### Example 3: Preparation of 1-(2,5-dimethoxy-4-(6,6,6-trifluorohexyl)phenyl)butan-2-amine (2a) hydrochloride

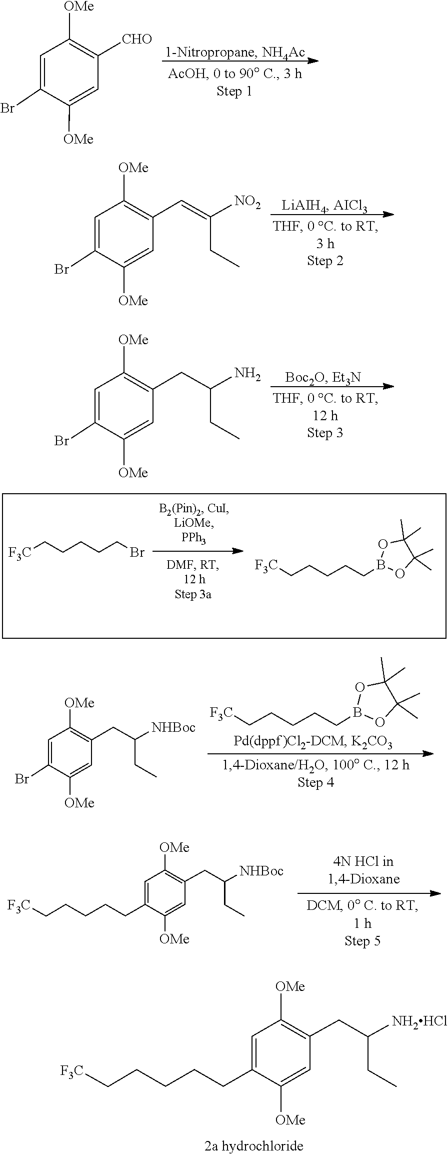

### Step 1: Preparation of (E)-1-bromo-2,5-dimethoxy-4-(2-nitrobut-1-en-1-yl)benzene

To a stirred solution of 4-bromo-2,5-dimethoxybenzaldehyde (10.0 g, 40.8 mmol) in 1-nitropropane (7.3 mL, 816 mmol) was added ammonium acetate (4.6 g, 61.2 mmol) at 0° C. The reaction mixture was then warmed to 90° C. and stirred for 3 h. The progress of the reaction was monitored by TLC. After completion, the reaction mixture was diluted with chilled water (100 mL) and extracted with ethyl acetate (250 mL×2). The combined organic extracts were washed with brine (100 mL), dried over anhydrous Na₂SO₄, filtered, and concentrated under reduced pressure to obtain the crude compound. This crude material was purified by flash column chromatography (silica gel; eluting with 0-10% ethyl acetate in heptanes) to afford the desired (E)-1-bromo-2,5-dimethoxy-4-(2-nitrobut-1-en-1-yl)benzene as a yellow solid (Yield: 5.2 g, 40%). ¹H NMR (400 MHz, CDCl₃) δ ppm=8.11 (s, 1H), 7.15 (s, 1H), 6.83 (s, 1H), 3.87 (s, 3H), 3.84 (s, 3H), 2.80 (q, J=7.2 Hz, 2H), 1.27 (t, J=7.2 Hz, 3H).

### Step 2: Preparation of 1-(4-bromo-2,5-dimethoxyphenyl)butan-2-amine

To a stirred solution of LAH (2.0 M in THF; 24.67 mL, 49.35 mmol) in anhydrous THF (100 mL) was added AlCl₃ (2.19 g, 16.45 mmol) portionwise at 0° C. under argon atmosphere. The reaction mixture was stirred at 0° C. for 30 min and (E)-1-bromo-5-methoxy-2-methyl-4-(2-nitrobut-1-en-1-yl)benzene (5.2 g, 16.45 mmol) in THF (30 mL) was added drop-wise over a period of 10 min at 0° C. The reaction mixture was warmed to room temperature and stirred for 3 h. Reaction progress was monitored by TLC. Upon completion, the reaction mixture was allowed to cool to 0° C. and THF/water (1:1, 50 mL) was added at the same temperature until effervescence stopped. The reaction mixture was then diluted with THF (50 mL) and stirred at room temperature for 30 min under N₂ atmosphere. The reaction mixture was filtered through a Celite pad and the filter cake was washed with THF (100 mL). The combined filtrates were dried over anhydrous Na₂SO₄, filtered, and concentrated under reduced pressure to afford 1-(4-bromo-2,5-dimethoxyphenyl)butan-2-amine as a gummy solid (Yield: 3.0 g, 63%), which was used directly for the next step.

### Step 3: Preparation of tert-butyl (1-(4-bromo-2,5-dimethoxyphenyl)butan-2-yl)carbamate

To a stirred solution of 1-(4-bromo-2,5-dimethoxyphenyl)butan-2-amine (3.0 g, 10.41 mmol) in THF (50 mL) was added triethylamine (4.5 mL, 31.23 mmol) followed by di-tert-butyl dicarbonate (4.78 mL, 20.82 mmol) at 0° C. The reaction mixture was then warmed to room temperature and stirred for 12 h. The progress of the reaction was monitored by TLC. Upon completion, the reaction mixture was diluted with cold water (50 mL) and extracted with ethyl acetate (75 mL×2). The combined organic extracts were washed with brine (50 mL), dried over anhydrous Na₂SO₄, filtered, and concentrated under reduced pressure to provide the crude compound. This crude material was purified by flash column chromatography (silica gel; eluting with 0-10% ethyl acetate in heptanes) to afford tert-butyl (1-(4-bromo-2,5-dimethoxyphenyl)butan-2-yl)carbamate as an off-white solid (Yield: 2.0 g, 49%). LC-MS m/z 288 [M+H−100]⁺; ¹H NMR (400 MHz, CDCl₃) δ ppm=7.02 (s, 1H), 6.75 (s, 1H), 4.47 (br s, 1H), 3.84 (s, 3H), 3.84 (s, 3H), 3.75 (s, 1H), 2.76-2.68 (m, 2H), 1.58-1.41 (m, 2H), 1.36 (s, 9H), 0.94 (t, J=7.2 Hz, 3H).

### Step 3a: Preparation of 4,4,5,5-tetramethyl-2-(6,6,6-trifluorohexyl)-1,3,2-dioxaborolane

To a solution of copper(I) iodide (261 mg, 1.37 mmol), triphenylphosphine (467 mg, 1.78 mmol), lithium methoxide (1.04 g, 27.4 mmol) and bis(pinacolato)diboron (5.22 g, 20.5 mmol) in degassed dimethylformamide (12 mL), was added 6-bromo-1,1,1-trifluorohexane (3 g, 13.7 mmol) in dimethylformamide (0.5 mL). The reaction mixture was stirred at room temperature for 12 h. The progress of the reaction was monitored by TLC. Upon completion, the reaction mixture was diluted with diethyl ether (50 mL) and filtered through a Celite pad. The filtrate was diluted with water (50 mL) and extracted with diethyl ether (50 mL×2). The combined organic layers were washed with brine (50 mL), dried over Na₂SO₄, filtered, and concentrated under reduced pressure to provide the crude product. This material was purified by flash column chromatography (silica gel; eluting with 0-10% ethyl acetate in heptanes) to afford 4,4,5,5-tetramethyl-2-(6,6,6-trifluorohexyl)-1,3,2-dioxaborolane as a colorless liquid (Yield: 2.5 g, 69%). ¹H NMR (400 MHz, CDCl₃) δ ppm=2.10-1.98 (m, 2H), 1.59-1.50 (m, 2H), 1.48-1.32 (m, 4H), 1.48-1.32 (m, 4H), 1.8-1.20 (m, 12H), 0.78 (t, J=8 Hz, 3H).

### Step 4: Preparation of tert-butyl (1-(2,5-dimethoxy-4-(6,6,6-trifluorohexyl)phenyl)butan-2-yl)carbamate

To a stirred solution of tert-butyl (1-(4-bromo-2,5-dimethoxyphenyl)butan-2-yl)carbamate (0.60 g, 1.94 mmol) in 1,4-dioxane/water (7:3, 6 mL) was added 4,4,5,5-tetramethyl-2-(6,6,6-trifluorohexyl)-1,3,2-dioxaborolane (1.64 g, 6.14 mmol), dipotassium carbonate (641 mg, 4.63 mmol) and [1,1′-bis(diphenylphosphino)ferrocene]dichloropalladium(II)-CH₂Cl₂ complex (63.12 mg, 77.28 μmol) under argon at room temperature. The reaction mixture was purged with argon for 10 min, then heated at 100° C. for 12 h. The progress of the reaction was monitored by LC-MS and TLC. Upon completion, the reaction mixture was diluted with ethyl acetate (20 mL) and filtered through a Celite pad. The filtrate was partitioned between ethyl acetate (10 mL) and water (10 mL). The organic layer was washed with brine (20 mL), dried over Na₂SO₄, filtered, and concentrated under reduced pressure. This crude material was purified by flash chromatography (silica gel; eluting with 0-15% ethyl acetate in heptanes) to afford tert-butyl (1-(2,5-dimethoxy-4-(6,6,6-trifluorohexyl)phenyl)butan-2-yl)carbamate as a yellow solid (Yield: 100 mg, 15%). LC-MS m/z 448.00 [M+H]⁺; ¹H NMR (400 MHz, CDCl₃) δ ppm=6.63 (d, J=7.6 Hz, 2H), 4.63 (br s, 1H), 3.78 (s, 3H), 3.77 (s, 3H), 3.76 (br s, 1H), 2.72-2.71 (m, 2H), 2.57 (t, J=8 Hz, 2H), 2.10-2.00 (m, 2H), 1.56-1.49 (m, 3H), 1.45-1.30 (m, 12H), 1.29-1.20 (m, 2H), 0.84 (t, J=8 Hz, 3H).

### Step 5: Preparation of 1-(2,5-dimethoxy-4-(6,6,6-trifluorohexyl)phenyl)butan-2-amine (2a) hydrochloride

To a stirred solution of tert-butyl (1-(2,5-dimethoxy-4-(6,6,6-trifluorohexyl)phenyl)butan-2-yl)carbamate (0.1 g, 0.223 mmol) in dichloromethane was added HCl solution (4.0 M in 1,4-dioxane; 0.56 mL, 2.23 mmol) at 0° C. After complete addition, the reaction mixture was warmed to room temperature and stirred for 1 h. The progress of the reaction was monitored by TLC and LC-MS. Upon completion, the reaction mixture was concentrated under reduced pressure and the residue was washed with diethyl ether (5 mL) to afford the desired 1-[2,5-dimethoxy-4-(6,6,6-trifluorohexyl)phenyl]butan-2-amine (2a) hydrochloride as an off-white solid (Yield: 57 mg, 66%). LC-MS m/z 348.30 [M+H]⁺; ¹H NMR (400 MHz, DMSO-d₆) δ ppm=7.81 (br s, 3H), 6.81 (s, 1H), 6.79 (s, 1H), 3.73 (s, 3H), 3.73 (s, 3H), 3.27-3.21 (m, 1H), 2.77 (d, J=6.8 Hz, 2H), 2.56-2.50 (m, 2H), 2.29-2.15 (m, 2H), 1.57-1.45 (m, 6H), 1.40-1.35 (m, 2H), 0.91 (t, J=7.8 Hz, 3H).

### Example 4: Preparation of 1-(4-(5,5-difluorohexyl)-2,5-dimethoxyphenyl)butan-2-amine (2b) hydrochloride

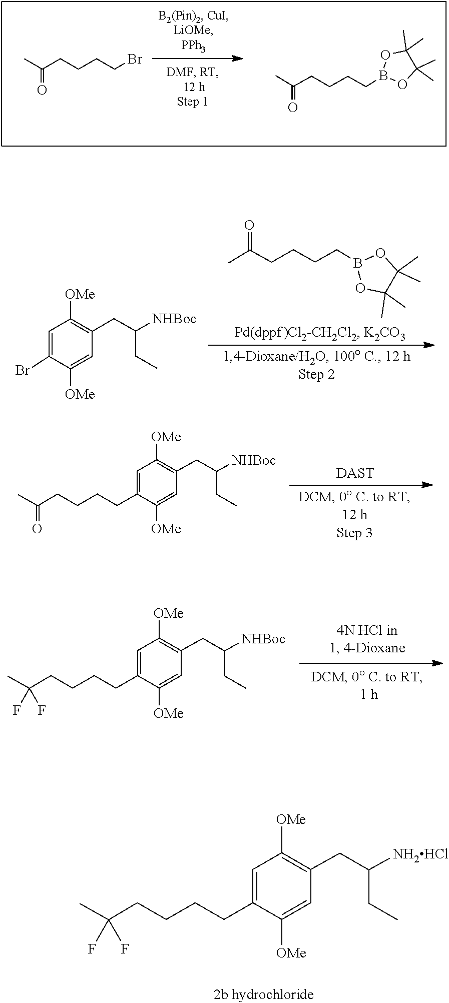

### Step 1: Preparation of 6-(4,4,5,5-tetramethyl-1,3,2-dioxaborolan-2-yl)hexan-2-one

To a solution of copper(I) iodide (319 mg, 1.68 mmol), triphenylphosphine (571 mg, 2.18 mmol), lithium methoxide (1.27 g, 33.6 mmol), and bis(pinacolato)diboron (3.0 g, 25.2 mmol) in degassed dimethylformamide (20 mL), was added 6-bromohexan-2-one (3 g, 16.8 mmol) in dimethylformamide (0.5 mL). The reaction mixture was stirred at room temperature for 12 h. The progress of the reaction was monitored by TLC. Upon completion, the reaction mixture was diluted with diethyl ether (50 mL) and filtered through a Celite pad. The filtrate was diluted with water (30 mL) and extracted with diethyl ether (50 mL×2). The combined organic layers were washed with brine (30 mL), dried over Na₂SO₄, filtered, and concentrated under reduced pressure to provide crude 6-(4,4,5,5-tetramethyl-1,3,2-dioxaborolan-2-yl)hexan-2-one as a colorless liquid (Yield: 2.0 g, 53%). This crude material was used directly for the next step without further purification.

### Step 2: Preparation of tert-butyl (1-(2,5-dimethoxy-4-(5-oxohexyl)phenyl)butan-2-yl)carbamate

To a stirred solution of tert-butyl (1-(4-bromo-2,5-dimethoxyphenyl)butan-2-yl)carbamate in 1,4-dioxane/water (7:3, 6 mL) was added 6-(4,4,5,5-tetramethyl-1,3,2-dioxaborolan-2-yl)hexan-2-one (1.02 g, 4.51 mmol), dipotassium carbonate (372 mg, 2.7 mmol), and [1,1′-bis(diphenylphosphino)ferrocene]dichloropalladium(II)-CH₂Cl₂ complex (29 mg, 0.036 mmol) under argon at room temperature. The reaction mixture was purged with argon for 10 min and then heated at 100° C. for 12 h. The progress of the reaction was monitored by LC-MS and TLC. Upon completion, the reaction mixture was diluted with ethyl acetate (20 mL) and filtered through a Celite pad. The filtrate was partitioned between ethyl acetate (10 mL) and water (10 mL). The combined organic layer was washed with brine (20 mL), dried over Na₂SO₄, filtered, and concentrated under reduced pressure. This crude material was purified by flash chromatography (silica gel; eluting with 0-15% ethyl acetate in heptanes) to afford tert-butyl (1-(2,5-dimethoxy-4-(5-oxohexyl)phenyl)butan-2-yl)carbamate as an off-white solid (Yield: 110 mg, 30%). LC-MS m/z 368.40 [M+H]⁺; ¹H NMR (400 MHz, CDCl₃) δ ppm=6.63 (s, 2H), 4.61 (br s, 1H), 3.77 (s, 3H), 3.76 (s, 3H), 3.76-3.70 (m, 1H), 2.71 (d, J=4.0 Hz, 2H), 2.57 (t, J=8.0 Hz, 2H), 2.45 (t, J=8.0 Hz, 2H), 2.12 (s, 3H), 1.65-1.61 (m, 2H), 1.59-1.50 (m, 2H), 1.36 (s, 9H), 1.28-1.23 (m, 2H), 0.93 (t, J=8.0 Hz, 3H).

### Step 3: Preparation of tert-butyl (1-(4-(5,5-difluorohexyl)-2,5-dimethoxyphenyl) butan-2-yl)carbamate

To a stirred solution of tert-butyl (1-(2,5-dimethoxy-4-(5-oxohexyl)phenyl)butan-2-yl)carbamate (110 mg, 0.27 mmol) in DCM (15 mL) was added diethylaminosulfur trifluoride (217 mg, 1.35 mmol) portionwise at 0° C. The reaction mixture was warmed to room temperature and stirred for 12 h. The progress of the reaction was monitored by LC-MS and TLC. Upon completion, the reaction mixture was diluted with chilled water (20 mL) and extracted with DCM (30 mL×2). The combined organic fractions were washed with brine (20 mL), dried over Na₂SO₄, filtered, and concentrated under reduced pressure. The resulting crude material was purified by flash chromatography (silica gel; eluting with 0-15% ethyl acetate in heptanes) to afford tert-butyl (1-(4-(5,5-difluorohexyl)-2,5-dimethoxyphenyl)butan-2-yl)carbamate as an off-white solid (Yield: 50 mg, 43%). LC-MS m/z 330 [M+H−100]⁺.

### Step 4: Preparation of 1-(4-(5,5-difluorohexyl)-2,5-dimethoxyphenyl)butan-2-amine (2b) hydrochloride

To a stirred solution of tert-butyl (1-(4-(5,5-difluorohexyl)-2,5-dimethoxyphenyl)butan-2-yl)carbamate (50 mg, 0.116 mmol) in dichloromethane (0.5 mL) was added HCl solution (4.0 M in 1,4-dioxane; 0.29 mL, 1.16 mmol) at 0° C. After complete addition, the reaction mixture was warmed to room temperature and stirred for 1 h. The progress of the reaction was monitored by TLC and LC-MS. Upon completion, the reaction mixture was concentrated under reduced pressure and the residue was washed with diethyl ether (5.0 mL) to afford 1-(4-(5,5-difluorohexyl)-2,5-dimethoxyphenyl)butan-2-amine (2b) hydrochloride as an off-white solid (Yield: 30 mg, 70%). LC-MS m/z 330.30 [M+H]⁺; 1H NMR (400 MHz, DMSO-d₆) δ ppm=7.83 (br s, 3H), 6.81 (s, 1H), 6.80 (s, 1H), 3.73 (s, 6H), 3.52-3.22 (m, 1H), 2.78 (d, J=6.8 Hz, 2H), 2.56-2.49 (m, 2H), 1.94-1.82 (m, 2H), 1.62-1.38 (m, 9H), 0.91 (t, J=7.6 Hz, 3H); ¹⁹F NMR (376 MHz, DMSO-d₆) δ ppm=−88.16.

### Example 5: Preparation of 1-(4-(hexyl-6,6,6-d₃)-2,5-dimethoxyphenyl)butan-2-amine (3a) hydrochloride

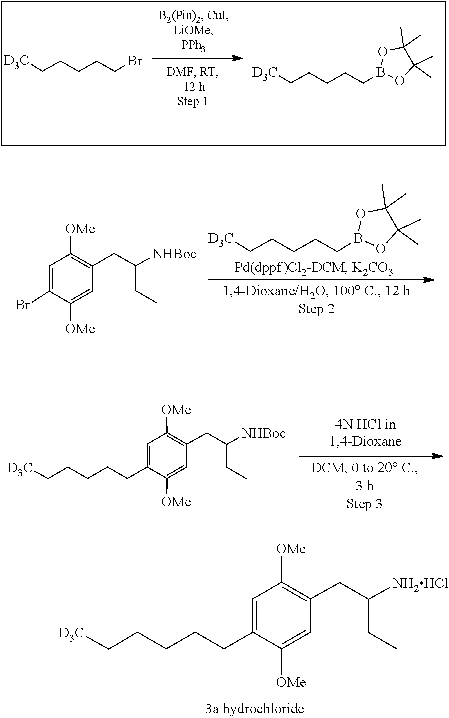

### Step 1: Preparation of 2-(hexyl-6,6,6-d3)-4,4,5,5-tetramethyl-1,3,2-dioxaborolane

To a solution of copper(I) iodide (90.6 mg, 0.476 mmol), triphenylphosphine (162 mg, 0.619 mmol), lithium methoxide (1.04 g, 27.4 mmol), and bis(pinacolato)diboron (1.81 g, 7.14 mmol) in degassed dimethylformamide (15 mL), was added a solution of 1-bromohexane-6,6,6-d₃ (3 g, 13.7 mmol) in dimethylformamide (0.5 mL). The reaction mixture was stirred at room temperature for 12 h. The progress of the reaction was monitored by TLC. Upon completion, the reaction mixture was diluted with diethyl ether (50 mL) and filtered through a Celite pad. The filtrate was diluted with water (50 mL) and extracted with diethyl ether (50 mL×2). The combined organic layers were washed with brine (50 mL), dried over Na₂SO₄, filtered, and concentrated under reduced pressure to afford crude 2-(hexyl-6,6,6-d₃)-4,4,5,5-tetramethyl-1,3,2-dioxaborolane as a colorless liquid (Yield: 0.6 g, 16%), which was used directly for the next step without further purification.

### Step 2: Preparation of tert-butyl (1-(4-(hexyl-6,6,6-d3)-2,5-dimethoxyphenyl)butan-2-yl)carbamate

To a stirred solution of tert-butyl (1-(4-bromo-2,5-dimethoxyphenyl)butan-2-yl)carbamate (0.4 g, 1.03 mmol) in 1,4-dioxane/water (7:3, 6 mL) was added 2-(hexyl-6,6,6-d₃)-4,4,5,5-tetramethyl-1,3,2-dioxaborolane (0.8 g, 4.12 mmol), dipotassium carbonate (427 mg, 3.03 mmol), and [1,1′-bis(diphenylphosphino)ferrocene]dichloropalladium(II)-CH₂Cl₂ complex (42 mg, 0.05 mmol) under argon at room temperature. The reaction mixture was purged with argon for 10 min, then heated at 100° C. for 12 h. The progress of the reaction was monitored by LC-MS and TLC. Upon completion, the reaction mixture was diluted with ethyl acetate (20 mL) and filtered through a Celite pad. The filtrate was partitioned between ethyl acetate (10 mL) and water (10 mL). The combined organic extracts were washed with brine (20 mL), dried over Na₂SO₄, filtered, and concentrated under reduced pressure. The resulting crude material was purified by flash chromatography (silica gel; eluting with 0-15% ethyl acetate in heptanes) to afford tert-butyl (1-(4-(hexyl-6,6,6-d3)-2,5-dimethoxyphenyl)butan-2-yl)carbamate as an off-white solid (Yield: 120 mg, 30%). LC-MS m/z 397.40 [M+H]⁺; ¹H NMR (400 MHz, CDCl₃) δ ppm=6.65 (s, 2H), 4.64 (d, J=7.6 Hz, 1H), 3.83 (s, 3H), 3.78 (s, 3H), 3.77-3.71 (m, 1H), 2.71 (d, J=6.0 Hz, 2H), 2.55 (t, J=8.0 Hz, 2H), 1.58-1.48 (m, 2H), 1.36 (s, 9H), 1.43-1.19 (m, 8H), 0.94 (t, J=8.0 Hz, 3H).

### Step 3: Preparation of 1-(4-(hexyl-6,6,6-d3)-2,5-dimethoxyphenyl)butan-2-amine (3a) hydrochloride

To a stirred solution of tert-butyl (1-(4-(hexyl-6,6,6-d3)-2,5-dimethoxyphenyl)butan-2-yl)carbamate (120 mg, 0.30 mmol) in dichloromethane (0.5 mL) was added HCl solution (4.0 M in 1,4-dioxane; 0.377 mL, 1.51 mmol) at 0° C. After complete addition, the reaction mixture was warmed to room temperature and stirred for 1 h. The progress of the reaction was monitored by TLC and LC-MS. Upon completion, the reaction mixture was concentrated under reduced pressure and the residue was washed with diethyl ether (5.0 mL) to afford 1-(4-(hexyl-6,6,6-d3)-2,5-dimethoxyphenyl)butan-2-amine (3a) hydrochloride as an off-white solid (Yield: 87 mg, 86%). LC-MS m/z 297.40 [M+H]⁺; ¹H NMR (400 MHz, DMSO-d₆) δ ppm=7.81 (s, 3H), 6.79 (s, 1H), 6.79 (s, 1H), 3.73 (s, 3H), 3.72 (s, 3H), 3.26-3.25 (m, 1H), 2.82-2.75 (m, 2H), 2.53-2.50 (m, 2H), 1.51-1.46 (m, 4H), 1.31-1.26 (m, 6H), 0.91 (t, J=8.0 Hz, 3H).

### Example 6: Serotonin Receptor Binding

The binding affinities of disclosed compounds at the ketanserin binding site of the 5-HT2A receptor, the LSD binding site of the 5-HT2B receptor, and the mesulergine binding site of the 5-HT2C receptor were determined in radioligand binding experiments.

Method. The affinity of the test compounds for 5-HT2A, 5-HT2B, and 5-HT2C receptors was determined in radioligand binding experiments with [³H]ketanserin, [³H]LSD, and [³H]mesulergine, respectively, by WuXi AppTec (Hong Kong) Limited, using methods adapted from the literature and under conditions described in Table 7.

TABLE 7
Assay conditions for radioligand binding assays.

| | 5-HT2A Binding | | 5-HT2B Binding | | 5-HT2C Binding | |
|---|---|---|---|---|---|---|
| Receptor | HEK293 stable | | HEK293 stable | | HEK293 stable | |
| Source | cell line | | cell line | | cell line | |
| Vehicle | 0.5% | DMSO | 0.5% | DMSO | 0.5% | DMSO |
| Incubation | 1 | h | 1 | h | 1 | h |
| Time | | | | | | |
| Incubation | 25 | °C | 25 | °C | 25 | °C |

| | | | |
|---|---|---|---|
| Temperature | | | |
| Incubation | 50 mM Tris-HCl, | 50 mM Tris-HCl, | 50 mM Tris-HCl, |
| Buffer | pH 7.4 | pH 7.4, 4 mM | 10 mM MgCl₂, |
| | | CaCl₂, 0.1% | 1 mM EDTA, |
| | | ascorbic acid | 0.1% BSA, |
| | | | pH 7.4 |
| Ligand | 1 nM | 1 nM | 2 nM |
| | [³H]ketanserin | [³H]LSD | [³H]mesulergine |
| Non-Specific | 1 μM | 50 μM | 1 μM |
| Ligand | ketanserin | serotonin | SB 206553 |

Results. Results of the radioligand binding assays are shown in Table 8. Both enantiomers of 1 had nearly identical binding at 5-HT2A receptors, but differed in their affinity for 5-HT2B and 5-HT2C receptors, with 1R having ~36-fold and ~7-fold lower affinity at 5-HT2B and 5-HT2C, respectively, and thus, greater selectivity for 5-HT2A. Compounds 2a and 2b were also potent ligands for 5-HT2A and 2a showed even higher selectivity than 1R for 5-HT2A over 5-HT2C.

TABLE 8
Results of receptor binding affinity experiments.

| | 5-HT2A Kᵢ (nM) | 5-HT2B Kᵢ (nM) | 5-HT2C Kᵢ (nM) |
|---|---|---|---|
| Compound | ([³H]ketanserin) | ([³H]LSD) | ([³H]mesulergine) |
| 1R | 3.44 | 111.37 | 18.01 |
| 1S | 3.34 | 3.10 | 2.68 |
| 2a | 5.40 | NT | 61.73 |
| 2b | 9.52 | NT | NT |

NT = not tested

### Example 7. Functional Activity at Serotonin Receptors

Disclosed compounds were tested for agonist activity at several serotonin receptor subtypes (5-HT2A, 2-HT2B, and 5-HT2C) using Ca²⁺ flux functional assays, with the results summarized in Table 9. Both enantiomers of 1 exhibited potent agonist activity at 5-HT2A, suggestive of possible therapeutic effects. Compound 1R was ~2-fold more potent at 5-HT2A and ~3-fold more potent at 5-HT2C compared to 1S. Efficacy at 5-HT2A was also greater for 1R compared to 1S. Both compounds showed very weak activity at 5-HT2B. Compounds 2a and 2b were substantially less potent than 1R and 1S in the 5-HT2A FLIPR assay despite similar binding affinity (see Example 6). Compound 2a was also substantially lower efficacy than 1R or 1S.

Test Compounds. Compounds were prepared as described above.

Functional Assays at 5-HT2A and 5-HT2B. Agonist activity at 5-HT2A and 5-HT2B receptors was determined using a FLIPR Ca²⁺ flux assay at WuXi AppTec (Hong Kong) Limited according to their standard protocols. Briefly, stably transfected cells expressing the receptor of interest (HEK293 for both 5-HT2A and 5-HT2B) were grown and plated in a 384-well plate and incubated at 37° C. and 5% CO₂ overnight. A solution of 250 mM probenecid in 1 mL FLIPR assay buffer was prepared fresh. This was combined with a fluorescent dye (Fluo-4 Direct™) to make a final assay concentration of 2.5 mM. Compounds were diluted 1:3.16 for 10 points and 750 nL was added to a 384 well compound plate using ECHO along with 30 μL assay buffer. The fluorescent dye was then added to the assay plate along with assay buffer to a final volume of 40 μL. The cell plate was incubated for 50 min at 37° C. and 5% CO₂ and placed into the FLIPR Tetra along with the compound plate. 10 μL of references and compounds were then transferred from the compound plate into the cell plate and the fluorescent signal was read.

Functional Assays at 5-HT2C. Agonist activity at 5-HT2C was determined using a FLIPR Ca²⁺ flux assay at Eurofins DiscoverX (Fremont, CA) according to their standard protocols. Briefly, stably transfected cells expressing the human 5-HT2C receptor were grown and plated in a 384-well plate and incubated at 37° C. and 5% CO₂ overnight. Assays were performed in 1× Dye Loading Buffer consisting of 1× Dye, 1× Additive A, and 2.5 mM Probenecid in HBSS/20 mM Hepes. Probenecid was prepared fresh. Cells were loaded with dye prior to testing and incubated at 37° C. for 30-60 minutes. After dye loading, cells were removed from the incubator and 10 μL HBSS/20 mM Hepes was added. 3× vehicle was included in the assay buffer. Cells were incubated for 30 mins at room temperature in the dark to equilibrate plate temperature. Intermediate dilution of sample stocks was performed to generate 4× sample in assay buffer. Compound agonist activity was measured on a FLIPR Tetra (MDS). Calcium mobilization was monitored for 2 minutes and 10 μL 4× sample in HBSS/20 mM Hepes was added to the cells 5 seconds into the assay.

TABLE 9
Agonist activity of compounds at select serotonin
receptors in Ca²⁺ flux functional assays.

| | | 5-HT2A | | 5-HT2B | | 5-HT2C |
|---|---|---|---|---|---|---|
| | 5-HT2A | % Act @ | 5-HT2B | % Act @ | 5-HT2C | % Act @ |
| | EC₅₀ | Max | EC₅₀ | Max | EC₅₀ | Max |
| Compound | (nM) | Dose | (nM) | Dose | (nM) | Dose |
| 1R | 23.1 | 76.81 | >10,000 | 14.84 | 1,191 | 104.29 |
| 1S | 43.77 | 59.74 | >10,000 | 16.06 | 3,127 | 106.27 |
| 2a | 156.1 | 36.23 | >10,000 | 4.85 | NT | NT |
| 2b | 5,056 | 71.34 | NT | NT | NT | NT |

NT = not tested

### Example 8: Functional Activity at the 5-HT2A Receptor in a Beta-Arrestin Recruitment Assay

Disclosed compounds were tested for agonist activity at the 5-HT2A receptor using a beta-arrestin (arrestin) recruitment functional assay, with the results summarized in Table 10. Both enantiomers of 1 were partial agonists in this assay and were highly potent, although 1R was substantially more efficacious compared to 1S. Compound 2a was higher efficacy in this assay than in the 5-HT2A FLIPR assay (see Example 7).

Arrestin Functional Assay at 5-HT2A. Recruitment of beta-arrestin was determined using a PathHunter assay at Eurofins DiscoverX (Fremont, CA) according to their standard protocols. PathHunter GPCR beta-arrestin assays take advantage of DiscoverX's proprietary Enzyme Fragment Complementation technology. The GPCR is fused in frame with a small enzyme donor fragment ProLink™ (PK) and co-expressed in cells stably expressing a fusion protein of beta-arrestin and the larger, N-terminal deletion mutant of beta-galactosidase. Activation of the GPCR (5-HT2A receptor in this case) stimulates binding of beta-arrestin to the PK-tagged GPCR and forces complementation of the two enzyme fragments, resulting in the formation of an active beta-galactosidase enzyme. This interaction leads to an increase in enzyme activity that can be measured using chemiluminescent PathHunter Detection Reagents. Briefly, PathHunter cells expressing 5-HT2A receptors were seeded in a volume of 20 μL into 384-well plates and incubated at 37° C. for the appropriate time prior to testing. For agonist activity determination, cells were incubated with 5 μL of 5× sample in assay buffer and incubated at 37° C. for 90-180 min with a vehicle concentration of 1%. Plates were then imaged on a microplate reader and the agonist activity was calculated using the following formula: % Activity=100%×(mean RLU of test sample−mean RLU of vehicle control)/(mean MAX control ligand−mean RLU of vehicle control), where RLU=relative light units.

TABLE 10
Agonist activity of compounds at the 5-HT2A
receptor in an arrestin recruitment assay.

| | 5-HT2A Arrestin | 5-HT2A Arrestin |
|---|---|---|
| Compound | EC₅₀ (nM) | % Act @ Max Dose |
| 1R | 6.229 | 82.46 |
| 1S | 7.846 | 58.55 |
| 2a | 33.22 | 56.84 |

### Example 9. Metabolic Stability in Human Liver Microsomes

Disclosed compounds were tested for stability in human liver microsomes (HLM), with the results summarized in Table 11. Compound 1R was substantially more stable than 1S in this system, as indicated by its lower CLᵢₙₜ and longer half-life, suggesting that 1R may have higher oral bioavailability than 1S in humans. Compounds 2a and 2b had significantly higher stability compared to 1R and 1S, whereas 3a showed intermediate stability.

Test Compounds. Compounds were prepared as described above.

HLM Stability. Pooled HLM from adult male and female donors (Corning 452117) were used. Microsomal incubations were carried out in multi-well plates. Liver microsomal incubation medium consisted of PBS (100 mM, pH 7.4), MgCl₂ (1 mM), and NADPH (1 mM), with 0.50 mg of liver microsomal protein per mL. Control incubations were performed by replacing the NADPH-cofactor system with PBS. Test compounds (1 μM, final solvent concentration 1.0%) were incubated with microsomes at 37° C. with constant shaking. Six time points over 60 minutes were analyzed, with 60 μL aliquots of the reaction mixture being drawn at each time point. The reaction aliquots were stopped by adding 180 μL of cold (4° C.) acetonitrile containing 200 ng/mL tolbutamide and 200 ng/mL labetalol as internal standards (IS), followed by shaking for 10 minutes, and then protein sedimentation by centrifugation at 4,000 rpm for 20 minutes at 4° C. Supernatant samples (80 μL) were diluted with water (240 μL) and analyzed for parent compound remaining using a fit-for-purpose liquid chromatography-tandem mass spectrometry (LC-MS/MS) method.

Data Analysis. The elimination constant (kₑₗ), half-life (t_{1/2}), and intrinsic clearance (CLᵢₙₜ) were determined in a plot of ln(AUC) versus time, using linear regression analysis.

TABLE 11
Intrinsic clearance (CLᵢₙₜ) and half-life
(t_{1/2}) of compounds in the presence of HLM.

| Compound | CLᵢₙₜ (μL/min/mg) | t_{1/2} (min) |
|---|---|---|
| 1R | 37.0 | 37.5 |
| 1S | 70.2 | 19.7 |
| 2a | <9.6 | >145 |
| 2b | <9.6 | >145 |
| 3a | 44.7 | 31 |

### Example 10. Metabolic Stability in Dog Liver Microsomes

Disclosed compounds were tested for stability in dog liver microsomes (DLM), with the results summarized in Table 12. Compound 1R was substantially less stable than 1S in this system, as indicated by its higher CLᵢₙₜ and shorter half-life, suggesting that 1R may have lower oral bioavailability than 1S in dogs. Consistent with the HLM results, 2a and 2b had significantly higher stability compared to 1R and 1S, whereas 3a showed modestly greater stability than 1R or 1S.

Test Compounds. Compounds were prepared as described above.

DLM Stability. Pooled DLM from adult male Beagle dogs (Xenotech D1000) were used. Microsomal incubations were carried out in multi-well plates. Liver microsomal incubation medium consisted of PBS (100 mM, pH 7.4), MgCl₂ (1 mM), and NADPH (1 mM), with 0.50 mg of liver microsomal protein per mL. Control incubations were performed by replacing the NADPH-cofactor system with PBS. Test compounds (1 μM, final solvent concentration 1.0%) were incubated with microsomes at 37° C. with constant shaking. Six time points over 60 minutes were analyzed, with 60 μL aliquots of the reaction mixture being drawn at each time point. The reaction aliquots were stopped by adding 180 μL of cold (4° C.) acetonitrile containing 200 ng/mL tolbutamide and 200 ng/mL labetalol as internal standards (IS), followed by shaking for 10 minutes, and then protein sedimentation by centrifugation at 4,000 rpm for 20 minutes at 4° C. Supernatant samples (80 μL) were diluted with water (240 μL) and analyzed for parent compound remaining using a fit-for-purpose liquid chromatography-tandem mass spectrometry (LC-MS/MS) method.

Data Analysis. The elimination constant (kₑₗ), half-life (t_{1/2}), and intrinsic clearance (CLᵢₙₜ) were determined in a plot of ln(AUC) versus time, using linear regression analysis.

TABLE 12
Intrinsic clearance (CLᵢₙₜ) and half-life
(t_{1/2}) of compounds in the presence of DLM.

| Compound | CLᵢₙₜ (μL/min/mg) | t_{1/2} (min) |
|---|---|---|
| 1R | 122.4 | 11.3 |
| 1S | 60.4 | 22.9 |
| 2a | 19.4 | 71.3 |
| 2b | 19.4 | 71.4 |
| 3a | 41.5 | 33.4 |

### Example 11. Oral Bioavailability in Rats

The pharmacokinetics of disclosed compounds were determined in the plasma of rats after oral and intravenous dosing, with the results presented in Table 13. In rats, 2a demonstrated improved absolute oral bioavailability (F), compared to 1.

Animals. Male Sprague Dawley rats were used in these studies. Animals were randomly assigned to treatment groups and were fasted overnight before oral dosing.

Drugs. The test compound was dissolved in 10% captisol (1) or 10% DMSO and 90% normal saline (2a) and administered intravenously (iv) at 2 mg/kg (1) or 1 mg/kg (2a) or orally (po) at 10 mg/kg (1) or 1 mg/kg (2a), calculated based on freebase, and at a volume of 10 mL/kg body weight for oral administration and 5 mL/kg for IV administration. The compounds were tested as the racemates.

Sample Collection and Bioanalysis. Blood samples were collected through the external jugular vein (via cannulation) at 0.083, 0.25, 0.5, 1, 2, 4, 8, and 24 h post-dose (total 8 time points/rat, 4 rats per administration route). At each time point, ~0.2 mL of blood was withdrawn and transferred into a pre-labeled 0.5-mL micro centrifuge tube containing 4 μL of 200 mM K₂EDTA solution as anticoagulant and mixed gently by inverting the tube to facilitate mixing of anticoagulant with the blood. Blood samples were kept on ice until centrifugation. The collected blood samples were centrifuged at 4,000 rpm for 10 min at 4° C. Plasma samples were separated after centrifugation, transferred into pre-labeled tubes, and stored at −70° C. until bioanalysis. For bioanalysis, a 200-μL aliquot of internal standard prepared in acetonitrile (cetirizine, 50 ng/mL) was added (except for blank, where 200 μL of acetonitrile was added) to a 96-well Solvinert filter plate followed by 5 μL of a study plasma sample or spiked calibration standard. The filter plate was then centrifuged for 5 minutes at a speed of 2,500 rpm at 4° C. with a 96-well sample collection plate. Following centrifugation, collected samples were analyzed using a fit-for-purpose LC-MS/MS method, with authentic samples of the analyte used for calibration and identification.

Data Analysis. Pharmacokinetic parameters were estimated using the non-compartmental analysis tool of Phoenix® WinNonlin software (Ver 8.1). The drug concentrations of samples below the lower limit of quantitation (LLOQ) were designated as zero.

TABLE 13
Selected pharmacokinetic parameters of disclosed
compounds in the plasma of Sprague Dawley rats.

| | | | AUC₀₋ₗₐₛₜ | AUC₀₋ₗₐₛₜ | t_{1/2} | t_{1/2} | |
|---|---|---|---|---|---|---|---|
| Compound | | C₀/Cₘₐₓ | (iv) | (po) | (iv) | (po) | F |
| Number | Dose | (ng/mL) | (ng*h/mL) | (ng*h/mL) | (h) | (h) | (%) |
| 1 | 2 | 389.49 | 354.46 | 137.79 | 2.16 | 2.63 | 8 |
| | mg/kg | (IV) | | | | | |
| | IV; | 35.12 | | | | | |
| | 10 | (PO) | | | | | |
| | mg/kg | | | | | | |
| | PO | | | | | | |
| 2a | 1 | 75.91 | 90.04 | 24.97 | 2.71 | 3.07 | 28 |
| | mg/kg | (IV) | | | | | |
| | IV | 29.68 | | | | | |
| | and | (PO) | | | | | |
| | PO | | | | | | |

While specific embodiments of the subject disclosure have been discussed, the above specification is illustrative and not restrictive. Many variations of the disclosure will become apparent to those skilled in the art upon review of this specification. The full scope of the disclosure should be determined by reference to the claims, along with their full scope of equivalents, and the specification, along with such variations.

1. A composition comprising a compound having the following R-enantiomeric structure: 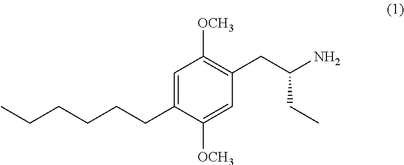 [SMILES: CCCCCCc1cc(OC)c(C[C@H](N)CC)cc1OC]
wherein the composition optionally includes the S-enantiomeric compound of Formula (1), provided that the R-enantiomeric compound of Formula (1) is in enantiomeric excess, or the compound of Formula (1) is in enantiomerically pure form in the composition;
or pharmaceutically acceptable salts of the compound.

2. The composition of claim 1, wherein the R-enantiomeric compound of Formula (1) is in mixture with the S-enantiomeric compound of Formula (1), provided that the R-enantiomeric compound of Formula (1) is present in an enantiomeric excess.

3. The composition of claim 1, wherein the composition contains the compound of Formula (1) in enantiomerically pure form.

4. A compound having the following structure: 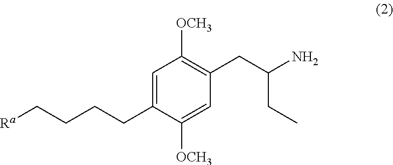
wherein:
R^{a} is selected from the group consisting of —CH₂CF₃, —CF₂CH₃, and —CF₂CF₃;
or pharmaceutically acceptable salts thereof.

5. The compound of claim 4, wherein the compound has the following structure: 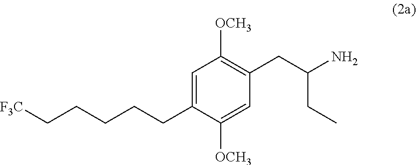 [SMILES: CCC(N)Cc1cc(OC)c(CCCCCC(F)(F)F)cc1OC]
or pharmaceutically acceptable salts thereof.

6. The compound of claim 4, wherein the compound has the following structure: 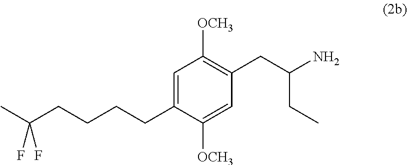 [SMILES: CCC(N)Cc1cc(OC)c(CCCCC(C)(F)F)cc1OC]
or pharmaceutically acceptable salts thereof.

7. The compound of claim 4, wherein the compound has the following structure: 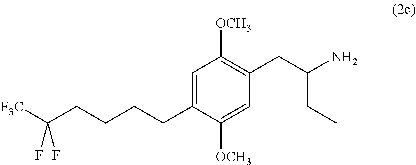 [SMILES: CCC(N)Cc1cc(OC)c(CCCCC(F)(F)C(F)(F)F)cc1OC]
or pharmaceutically acceptable salts thereof.

8. A compound having the following structure: 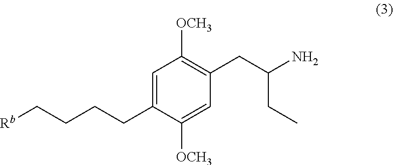
wherein:
R^{b} is —CH₂CD₃ or -CD₂CD₃;
or pharmaceutically acceptable salts thereof.

9. The compound of claim 8, wherein the compound has the following structure: 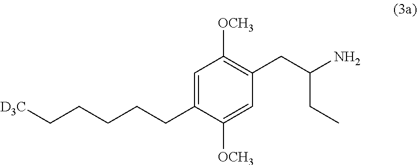 [SMILES: [2H]C([2H])([2H])CCCCCc1cc(OC)c(CC(N)CC)cc1OC]
or pharmaceutically acceptable salts thereof.

10. The compound of claim 8, wherein the compound has the following structure: 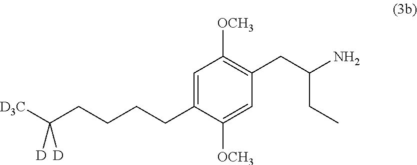 [SMILES: [2H]C([2H])([2H])C([2H])([2H])CCCCc1cc(OC)c(CC(N)CC)cc1OC]
or pharmaceutically acceptable salts thereof.

11. The compound of claim 8 or pharmaceutically acceptable salts thereof, wherein one or more of the indicated D is present in greater than the natural abundance.

12. The compound of claim 11 or pharmaceutically acceptable salts thereof, wherein one or more of the indicated D is at least or greater than 50% enriched.

13. The compound of claim 11 or pharmaceutically acceptable salts thereof, wherein one or more of the indicated D is at least or greater than 90% enriched.

14. The compound of claim 11 or pharmaceutically acceptable salts thereof, wherein one or more of the indicated D is at least or greater than 95% enriched.

15. A pharmaceutical composition comprising the composition of claim 1 and a pharmaceutically acceptable carrier therefor.

16. A method of treating a malady selected from the group consisting of (a) a psychiatric disease or disorder; (b) a headache or headache disorder; (c) an inflammatory disease or disorder; and (d) high intraocular pressure; comprising administering to a patient in need thereof a therapeutically effective amount of the composition of claim 1.

17.-30. (canceled)

31. A pharmaceutical composition comprising the compound of claim 4 or pharmaceutically acceptable salts thereof and a pharmaceutically acceptable carrier therefor.

32. A method of treating a malady selected from the group consisting of (a) a psychiatric disease or disorder; (b) a headache or headache disorder; (c) an inflammatory disease or disorder; and (d) high intraocular pressure; comprising administering to a patient in need thereof a therapeutically effective amount of the compound of claim 4 or pharmaceutically acceptable salts thereof.

33. A pharmaceutical composition comprising the compound of claim 8 or pharmaceutically acceptable salts thereof and a pharmaceutically acceptable carrier therefor.

34. A method of treating a malady selected from the group consisting of (a) a psychiatric disease or disorder; (b) a headache or headache disorder; (c) an inflammatory disease or disorder; and (d) high intraocular pressure; comprising administering to a patient in need thereof a therapeutically effective amount of the compound of claim 8 or pharmaceutically acceptable salts thereof.
